(19) United States
(12) Patent Application Publication
(10) Pub. No.: US 2022/0409282 A1
(43) Pub. Date: Dec. 29, 2022

(54) METHODS AND SYSTEMS FOR ASSISTING A USER IN POSITIONING AN AUTOMATED MEDICAL DEVICE RELATIVE TO A BODY OF A PATIENT

(71) Applicant: XACT ROBOTICS LTD., Caesarea (IL)

(72) Inventors: Moran SHOCHAT, Zikron Ya'aqov (IL); Ido ROTH, Pardes Hanna (IL); Dvir KRAUSHAR, Atlit (IL)

(21) Appl. No.: 17/780,991

(22) PCT Filed: Dec. 3, 2020
(86) PCT No.: PCT/IL2020/051247

§ 371 Date: May 29, 2022

Related U.S. Application Data
(60) Provisional application No. 62/944,048, filed on Dec. 5, 2019.

(51) Int. Cl. A61B 34/10; A61B 34/00; A61B 90/50; G06T 11/00

(52) U.S. Cl. CPC A61B 34/10; A61B 34/25; A61B 90/50; G06T 11/00; A61B 2034/107; A61B 2034/104; A61B 2034/252; G06T 2210/41

(57) ABSTRACT

Provided are systems, devices and methods for assisting a user in positioning an automated medical device on, or in close proximity to, a body of a subject, by simulating the position and orientation of the medical device on one or more images of the subject, and providing the user with instructions regarding the actual positioning of the medical device and/or correction thereof, based on the simulated position and orientation.

### TECHNICAL FIELD

The present disclosure relates to the field of medical procedures, and specifically, to methods, devices and systems for assisting and guiding a user in positioning an automated medical device relative to a body of a subject.

### BACKGROUND

Various diagnostic and therapeutic procedures used in clinical practice involve the insertion of medical instruments, such as needles and catheters, percutaneously to a subject's body, and in many cases further involve the steering of the medical instruments within the body, to reach the target region. The target region can be any internal body region, including, a lesion, tumor, organ or vessel. Examples of procedures requiring insertion and steering of such medical instruments include vaccinations, blood/fluid sampling, regional anesthesia, tissue biopsy, catheter insertion, cryogenic ablation, electrolytic ablation, fluid delivery, fluid drainage, brachytherapy, neurosurgery, deep brain stimulation, various minimally invasive surgeries, and the like.

The guidance and steering of medical tools, such as needles, in soft tissue is a complicated task that requires good three-dimensional coordination, knowledge of the patient's anatomy and a high level of experience, thus the use of automated (e.g., robotic) systems for performing these functions has been increasingly growing in recent years. Some automated systems are based on manipulating robotic arms and some utilize a body-mountable robotic device. Automated systems typically assist the physician in selecting an insertion point and in aligning the medical instrument with the insertion point and with the target. More advance systems also automatically insert and/or steer the instrument towards the target.

Accurately positioning and/or orienting the automated device on and/or relative to the patient's body such that the medical instrument can successfully reach the target can be challenging, especially when the target is located in a region which is difficult to navigate to, such as the liver dome. In such cases, proper positioning of the automated device may require several iterations, with a scan to register the device to the image space having to be initiated between each two consecutive iterations, to verify the position of the device. Multiple iterations and scanning are not only time consuming, but they also result in increased radiation exposure to both the medical staff and the patient. In addition, since the positioning of the automated device is based on estimated measurements of the physician (or another member of the medical staff), the final position and/or orientation of the device might still deviate from its required position and orientation.

Thus, there is a need for systems and methods for assisting and guiding a user (e.g., physician) in positioning and/or orienting a medical device relative to the patient's body.

The disclosures of each of the publications mentioned in this section and in other sections of the specification, are hereby incorporated by reference, each in its entirety.

### SUMMARY

According to some embodiments, the present disclosure is directed to systems, devices and methods for assisting a user (such as a healthcare provider) in positioning a medical device on a body of a subject, or in close proximity thereto, by simulating the position and orientation of the medical device relative to the body of the subject on one or more images of the subject, using a virtual medical device. The systems, devices and methods further allow providing the user with instructions regarding the actual (physical) positioning of the medical device and/or correction thereof, based on the simulated position and orientation.

According to some embodiments, the systems, devices and methods for assisting the user in positioning an automated medical device, as disclosed herein, are advantageous as they ultimately allow a safer, more efficient and more accurate medical procedure, whereby the positioning simulation and the guiding of the user as to the positioning of the actual medical device relative to the subject's body according to the simulations, are executed for each specific medical procedure and for each specific subject intended to undergo the medical procedure.

According to some embodiments, there is provided a method of assisting a user in positioning an automated medical device on, or in close proximity to, a body of a subject, the method may include:
displaying one or more images of a region of interest in the body of the subject;
defining on the one or more images a target and an entry point;
calculating a trajectory for a medical instrument from the entry point to the target;
simulating on the one or more images a position and an orientation of the automated medical device on, or in close proximity to, the body of the subject;
determining if the simulated position and orientation of the automated medical device is valid; and
if the simulated position and orientation of the automated medical device and the calculated trajectory are determined to be valid, providing positioning instructions to the user as to the positioning of the automated medical device on, or in close proximity to, the body of the subject.

According to some embodiments, if the simulated position and orientation of the automated medical device are determined to be invalid, the method may include prompting the user to define a new entry point and/or a new target on the one or more images.

According to some embodiments, if the simulated position and orientation of the automated medical device are determined to be invalid, the method may include simulating on the one or more images one or more additional positions and orientations of the automated medical device and determining if at least one of the one or more additional simulated positions and orientations of the automated medical device is valid.

According to some embodiments, if the simulated position and orientation of the automated medical device are determined to be invalid, the method may include recommending to the user an alternative simulated position and orientation of the automated medical device.

According to some embodiments, determining if the simulated position and orientation of the automated medical device are valid may include determining if the simulated position and orientation ensure alignment of a tip of the medical instrument with the entry point at an insertion angle that enables inserting and steering of the medical instrument according to the calculated trajectory, from the entry point to the target.

According to some embodiments, determining if the simulated position and orientation of the automated medical device are valid may include determining if, for the simulated position and orientation of the automated medical device, the calculated trajectory is valid.

According to some embodiments, determining if the calculated trajectory is valid may include determining if a curvature of the calculated trajectory exceeds a predetermined threshold.

According to some embodiments, determining if the simulated position and orientation of the automated medical device are valid may include determining if the rotation angles required from an end effector of the automated medical device are within a feasible rotation range for the end effector.

According to some embodiments, the method may include defining on the one or more images one or more obstacles to be avoided by the medical instrument.

According to some embodiments, simulating a position and an orientation of the automated medical device on the one or more images may include displaying a virtual medical device on the one or more images.

According to some embodiments, simulating a position and an orientation of the automated medical device on the one or more images may be executed automatically by means of at least one processor.

According to some embodiments, automatically simulating a position and an orientation of the automated medical device may be executed using image processing techniques (methods).

According to some embodiments, automatically simulating a position and an orientation of the automated medical device may be executed using one or more machine learning and/or deep learning algorithms.

According to some embodiments, simulating a position and an orientation of the automated medical device may include receiving user input regarding a position and an orientation of a virtual medical device displayed on the one or more images.

According to some embodiments, simulating a position and an orientation of the automated medical device may include rotating the one or more images, to simulate at least one alternative patient pose, and simulating a position and an orientation of the automated medical device on the at least one alternative patient pose.

According to some embodiments, the method may include adjusting the simulated position and orientation of the automated medical device. The adjustment may be carried out automatically by a processor, or manually by the user. According to some embodiments, the method may include receiving user input regarding the adjustment of the simulated position and orientation of the automated medical device.

According to some embodiments, providing positioning instructions to the user may include at least one of displaying the positioning instructions on a monitor and providing audio positioning instructions.

According to some embodiments, the positioning instructions provided to the user may include instructions to move, rotate, elevate or tilt the automated medical device, or a combination thereof.

According to some embodiments, providing positioning instructions to the user may include providing positioning instructions as to the positioning of an attachment apparatus configured for securing to the body of the subject and for coupling the automated medical device thereto, on the body of the subject.

According to some embodiments, the automated medical device is an automated insertion device configured to insert and steer the medical instrument toward the target according to the calculated trajectory.

According to some embodiments, there is provided a system for assisting a user in positioning an automated medical device on, or in close proximity to, a body of a subject, the system may include:

at least one processor configured to execute the method of assisting a user in positioning an automated medical device, as disclosed herein; and

a monitor configured to display at least one of: one or more images, a calculated trajectory and a simulated position and orientation of the automated medical device.

According to some embodiments, the system may include a user interface.

According to some embodiments, the automated medical device is configured to be attached to the body of the subject using an attachment apparatus.

According to some embodiments, the attachment apparatus may include one or more of a parallel lifting member configured to elevate the automated medical device relative to a surface of the body of the patient, and an angular lifting member configured to tilt the automated medical device relative to a surface of the body of the patient.

According to some embodiments, the system may include an auxiliary positioning mechanism configured to assist the user in at least one of positioning and orienting the automated medical device based on the provided positioning instructions.

According to some embodiments, the auxiliary positioning mechanism may include an orienting member configured to simulate one or more of the medical instrument and an end effector of the automated medical device.

According to some embodiments, the auxiliary positioning mechanism may include an electromechanical mechanism and it may be configured to be controlled by the processor to automatically execute the positioning instructions.

According to some embodiments, the auxiliary positioning mechanism may be a stand-alone device.

According to some embodiments, the auxiliary positioning mechanism may be part of, or couplable to, an attachment apparatus or an aiming apparatus, the attachment apparatus being configured for securing to the body of the patient and for receiving the automated medical device thereon, and the aiming apparatus being configured for removably coupling to the attachment apparatus and for assisting in ensuring alignment between a tip of the medical instrument and an entry point marked on the body of the patient.

According to some embodiments, there is provided a method of assisting a user in positioning an automated medical device on, or in close proximity to, a body of a subject, the method may include:

displaying one or more first images of a region of interest in the body of the subject, the one or more images showing one or more of the automated medical device and an attachment apparatus configured to be secured to the body of the patient and to receive the automated medical device thereon, positioned on the body of the subject at an initial position and orientation;

defining on the one or more images a target and an entry point;

calculating a trajectory for the medical instrument from the entry point to the target;

simulating on the one or more images a position and an orientation of the automated medical device on, or in close proximity to, the body of the subject;

comparing the actual position and orientation of the one or more of the automated medical device and the attachment frame to the simulated position and orientation of the automated medical device;

if the actual position and orientation of the one or more of the automated medical device and the attachment frame deviate from the simulated position and orientation of the automated medical device, providing correction instructions to the user as to the required correction to the actual position and orientation of the one or more of the automated medical device and the attachment frame;

displaying one or more second images of the region of interest, the one or more second images showing the one or more of the automated medical device and the attachment frame positioned on the body of the subject at a corrected position and orientation;

determining if the corrected position and orientation of the one or more of the automated medical device and the attachment frame are valid; and

if the corrected position and orientation of the one or more of the automated medical device and the attachment frame are determined to be valid, notifying the user.

According to some embodiments, if the corrected position and orientation of the one or more of the automated medical device and the attachment frame are determined to be invalid, the method may include simulating a new position and orientation of the automated medical device on, or in close proximity to, the body of the subject.

According to some embodiments, the method may include determining if the simulated position and orientation of the medical device is valid, prior to comparing the actual position and orientation of the one or more of the automated medical device and the attachment frame to the simulated position and orientation of the automated medical device.

According to some embodiments, determining if the simulated position and orientation of the medical device are valid may include determining if the simulated position and orientation ensure alignment of the tip of the medical instrument with the entry point at an insertion angle that enables inserting and steering of the medical instrument according to the calculated trajectory.

According to some embodiments, determining if the corrected position and orientation of the one or more of the automated medical device and the attachment frame are valid may include determining if the corrected position and orientation ensure alignment of the tip of the medical instrument with the entry point at an insertion angle that enables inserting and steering of the medical instrument according to the calculated trajectory.

According to some embodiments, there is provide a system for assisting a user in positioning an automated medical device on, or in close proximity to, a body of a subject, the system may include: at least one processor configured to execute the method of assisting a user in positioning an automated medical device on, or in close proximity to, a body of a subject, as disclosed herein; and a monitor configured to display at least one of: one or more images, a calculated trajectory and a simulated position and orientation of the automated medical device.

According to some embodiments, the system may include a user interface.

According to some embodiments, the system may include an auxiliary positioning mechanism configured to assist the user in executing the provided correction instructions.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not to scale.

FIG. 1 shows a schematic diagram of a system for inserting a medical instrument into the body of a subject, according to some embodiments;

FIGS. 2A-2B show perspective views of an exemplary device (FIG. 2A) and an exemplary control unit (FIG. 2B) for inserting and steering a medical instrument into the body of a subject, according to some embodiments;

FIG. 3 shows a flowchart showing the steps in a method for assisting the user in positioning an automated medical device on, or in close proximity to, the body of a subject, according to some embodiments;

FIG. 4A shows CT images of a subject showing an exemplary calculated trajectory from an entry point to a target in the subject's body, according to some embodiments;

FIG. 4B shows a virtual automated medical device displayed on CT images of a subject , according to some embodiments;

FIG. 4C shows an exemplary simulation of the position and the orientation of an automated medical device displayed on CT images of a subject, according to some embodiments;

FIG. 4D shows an exemplary simulation of the position and the orientation of an automated medical device, in which a virtual registration element penetrates the body of the subject, according to some embodiments;

FIG. 4E shows an exemplary adjusted simulation of the position and the orientation of an automated medical device displayed on CT images of a subject, according to some embodiments;

FIG. 4F shows the exemplary adjusted simulation of the position and the orientation of the automated medical device with the exemplary calculated trajectory displayed on CT images of a subject, according to some embodiments;

FIG. 5 shows an exemplary attachment apparatus configured for use with a body-mountable medical device, according to some embodiments;

FIG. 6A shows a rear view of an exemplary medical device and an exemplary attachment apparatus, prior to coupling, according to some embodiments;

FIG. 6B shows the exemplary medical device positioned on the exemplary attachment frame, according to some embodiment;

FIG. 7 shows an exemplary aiming apparatus coupled to an attachment frame, according to some embodiments;

FIGS. 8A-8B show an exemplary aiming apparatus having an orienting mechanism, according to some embodiments;

FIG. 9 shows a flowchart showing the steps in a method for assisting the user in positioning an automated medical device on, or in close proximity to, the body of a subject, according to some embodiments;

FIGS. 10A-10B show CT images of a subject and schematic illustrations of exemplary instructions given to a user regarding corrections to the positioning of an attachment frame on a body of a subject;

FIG. 11 shows a flowchart showing the steps in a method for assisting the user in positioning an automated medical device on, or in close proximity to, the body of a subject, according to some embodiments.

### DETAILED DESCRIPTION

The principles, uses, and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

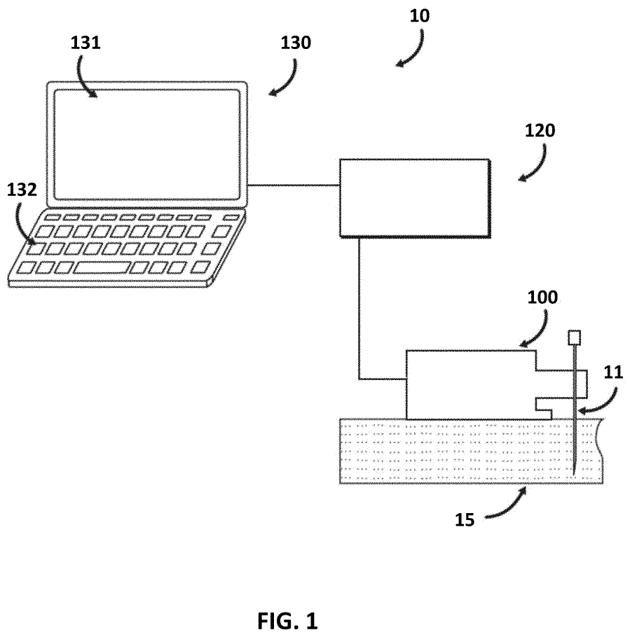

FIG. 1 shows a schematic diagram of a system 10 for inserting a medical instrument (e.g., needle or introducer) into a subject's body. The system 10 includes an automated medical device, for example automated insertion device 100, which may be configured for inserting and steering an instrument 11 toward the target in the subject's body 15. The instrument 11 may be removably couplable to the insertion device 100, such that the insertion device 100 can be used repeatedly with new instruments. In some embodiments, the automated device is a disposable device, i.e., a device which is intended to be disposed of after a single use. In some embodiments, the medical instruments are disposable. In some embodiments, the medical instruments are reusable.

The system 10 may include, or be configured to operate in conjunction with, an imaging system, such that the insertion procedure is image-guided. The utilized imaging modality may be any one of X-ray fluoroscopy, CT, cone beam CT, CT fluoroscopy, MRI, ultrasound, or any other suitable imaging modality.

In some embodiments, the insertion device 100 may be a robotic device configured for mounting on the subject's body 15, as shown in FIG. 1. In other embodiments, the insertion device 100 may be configured as a robotic arm or as a robotic device configured for positioning on the subject's body, or in close proximity thereto, using a dedicated arm or base, which is secured to the patient's bed, to a cart positioned adjacent to the patient bed or to the imaging device, as described, for example, in U.S. Pat. Nos. 10,507,067 and 10,639,107, both to Glozman et al, and both incorporated herein by reference in their entireties.

The system 10 may further include a controller 120, e.g., a robot controller, which controls the movement of the insertion device 100 and the steering of the medical instrument 11 towards the target (e.g., lesion or tumor) within the subject's body 15. In some embodiments, the controller 120 may be further configured to control the operation of sensors (not shown), such as a force sensor and/or an acceleration sensor, implemented in the system 10. Use of sensor/s for sensing parameters associated with the interaction between a medical instrument and a bodily tissue, e.g., a force sensor, and utilizing the sensor data for guiding the insertion of the instrument and/or for initiating imaging, is described, for example, in co-owned U.S. Patent Application Publication No. 2018/250,078, to Shochat et al, which is incorporated herein by reference in its entirety. The controller 120 may be a separate component, as shown in FIG. 1. Alternatively, at least a portion of the controller 120 may be embedded within the insertion device 100, and/or within the computer 130 of the system 10. Computer 130 may include one or more processors (not shown) configured for image processing, calculation of the optimal insertion trajectory, etc., and a display/monitor 131 on which images obtained using an imaging system, or image-views created from a set of images, the calculated insertion trajectory, etc., can be displayed. The computer 130 may be a personal computer (PC), a laptop, a tablet, a mobile telephone or any other processor-based device. The computer 130 may also include a user interface 132, which may be in the form of buttons, switches, keys, keyboard, computer mouse, joystick, touch-sensitive screen, etc. The display 131 and user interface 132 may be two separate components, or they may form together a single component, such as a touch-sensitive screen (“touch screen”). In some embodiments, the user may operate the insertion device using a pedal or an activation button. In some embodiments, the system may include a remote control unit (not shown), which may enable the user to activate the insertion device from a remote location, such as the control room adjacent the procedure room, a different location at the medical facility or a location outside the medical facility.

The computer 130 may be configured, inter alia, to receive, process and visualize on the monitor 131 images obtained from the imaging system (in DICOM format, for example), to calculate the optimal trajectory for the medical instrument, and to control steering of the instrument toward the target. In some embodiments, steering of the instrument may be executed in a closed-loop manner, i.e., the processor may generate motion commands to the insertion device 100 via a controller 120 and receive feedback regarding the actual location of the instrument, which is then used for real-time trajectory corrections, as disclosed, for example, in U.S. Pat. No. 8,348,861, to Glozman et al, which is incorporated herein by reference in its entirety. In some embodiments, the optimal trajectory may be calculated based on input from the user, such as the target, entry point and, optionally, areas to avoid en route (also referred to as “obstacles”), which the user may mark on at least one of the obtained images (or on an image-view generated from a set of images). In other embodiments, the processor may be further configured to automatically identify and mark one or more of the target, the obstacles and the optimal entry point. The optimal trajectory may be calculated in a two-dimensional plane or in a three-dimensional space, as described, for example, in abovementioned U.S. Pat. No. 8,348,861 and in co-owned International patent Application No. PCT/IL2020/051219, which is incorporated herein by reference in its entirety.

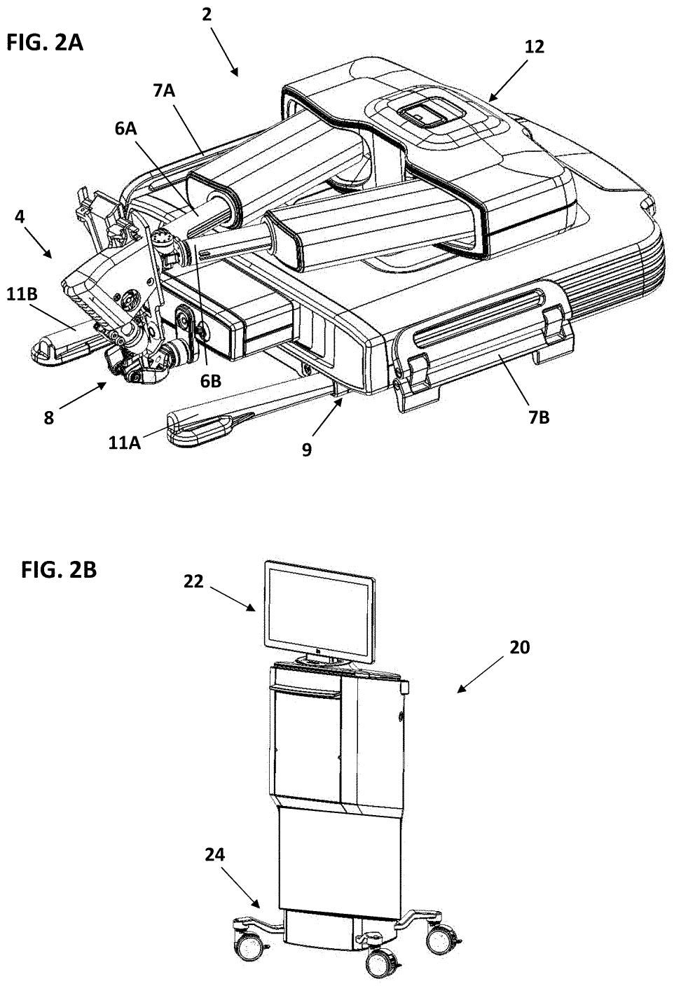

Reference is now made to FIG. 2A, which shows a schematic perspective view of an exemplary automated (i.e., robotic) device for inserting and steering a medical instrument in the subject's body. As shown in FIG. 2A, the insertion and steering device 2 may include a housing (also referred to as “cover”) 12 accommodating therein at least a portion of the steering mechanism. The steering mechanism may include movable platforms (not shown) and movable arms 6A and 6B, configured to allow or control movement of an end effector (also referred to as “control head”) 4, at any one of desired movement angles or axis, as disclosed, for example, in co-owned U.S. Patent Application Publication No. 2019/290,372, to Arnold et al. To the end 8 of control head 4, a suitable medical instrument (not shown) may be connected, either directly or by means of a suitable insertion module, such as the insertion module disclosed in co-owned U.S. Patent Application Publication No. 2017/258,489, to Galili et al, which is incorporated herein by reference in its entirety. In some embodiments, the medical instrument may be removably coupled to control head 4, such that device 2 can be used repeatedly with new medical instruments. The medical instrument may be either disposable or reusable, and it may be any suitable instrument capable of being inserted and steered within the body of the subject, to reach a designated target, wherein the control of the operation and movement of the medical instrument is effected by control head 4. Control head 4 may be controlled by a suitable control system, as detailed herein.

According to some embodiments, the medical instrument may be selected from, but not limited to: a needle, a probe (e.g., an ablation probe), a port, an introducer, a catheter (e.g., a drainage needle catheter), a cannula, a surgical tool, a fluid delivery tool, or any other suitable insertable tool configured to be inserted into a subject's body for diagnostic and/or therapeutic purposes. In some embodiments, the medical instrument includes a tip at the distal end thereof (i.e., the end which is inserted into the subject's body).

In some embodiments, the device 2 may have a plurality of degrees of freedom (DOF) in operating and controlling the movement the of the medical instrument, via the end effector to which the medical instrument may be coupled, along one or more axis. For example, the device may have up to six degrees of freedom. For example, the device may have at least five degrees of freedom. For example, the device may have five degrees of freedom, including: forward-backward and left-right linear translations, front-back and left-right rotations, and longitudinal translation toward the subject's body (insertion). In some embodiments, the device may have six degrees of freedom, which may include the five degrees of freedom described above and, in addition, rotation of the medical instrument about its longitudinal axis.

In some embodiments, the device 2 may further include a base 9, which allows positioning of the device on or in close proximity to the subject's body. In some embodiments, the device may be attached to the subject's body, either directly or via a suitable mounting unit, such as an attachment apparatus (also referred to as “attachment frame” or “mediator plate”), which will be described hereinbelow. Attachment of the device 2 to an attachment frame may be carried out using dedicated latches, such as latches 7A and 7B. In some embodiments, the device 2 may be coupled to a dedicated arm or base which is secured to the patient's bed, to a cart positioned adjacent the patient's bed or to an imaging device, and held on the subject's body or in close proximity thereto.

In some embodiments, the device 2 includes electronic components and motors (not shown) allowing the controlled operation of the device 2 in inserting and steering the medical instrument. In some embodiments, the housing 12 is configured to cover and protect the mechanical and electronic components of the device 2 from being damaged or otherwise compromised. In some embodiments, the housing 12 may include at least one adjustable cover, and it may be configured to protect the device from being soiled by dirt, as well as by blood and/or other bodily fluids, thus preventing/minimizing the risk of cross-contamination between patients, as disclosed, for example, in co-owned International Patent Application No. PCT/IL2020/051220, which is incorporated herein by reference in its entirety.

In some exemplary embodiments, the device may further include fiducial markers (also referred to as “registration elements”) disposed at specific locations on the device 2, such as registration elements 10A and 10B, for registration of the device 2 to the image space, in image-guided procedures.

In some embodiments, the device 2 is part of a system for inserting and steering a medical instrument in a subject's body based on a preplanned and real-time updated 3D trajectory of a tip of the medical instrument, as disclosed, for example, in abovementioned International Application No. PCT/IL2020/051219. In some embodiments, the system includes the steering and insertion device 2, as disclosed herein, and a control unit (or—“workstation” or “console”) 20 configured to allow control of the operating parameters of the device 2, as shown in FIG. 2B.

Reference is now made to FIG. 2B, which shows a perspective view of an exemplary workstation 20. The workstation 20 may include a display 22 and a user interface (not shown). The monitor and user interface may be two separate components, or they may form together a single component (e.g., in the form of a touch-screen). The workstation 20 may include one or more suitable processors (for example, in the form of a PC) and one or more suitable controllers, configured to physically and/or functionally interact with the insertion and steering device, to determine and control the operation thereof. The workstation may be portable (e.g., by having or being placed on a movable platform 24).

In some embodiments, the processor (for example, as part of a computer) may be configured to perform one or more of: determine (plan) a trajectory (pathway) for the medical instrument to reach the target; update the trajectory in real-time, for example due to movement of the target from its initial identified position as a result of the advancement of the medical instrument within the patient's body; present the planned and/or updated trajectory; control the movement (steering and insertion) of the medical instrument based on the planned and/or updated trajectory, by providing executable instructions (directly or via the one or more controllers) to the device; determine the actual location of the tip of medical instrument by performing required compensation calculations; receive, process and visualize on the monitor images obtained from the imaging system, or image-views created from a set of images; and the like, or any combination thereof.

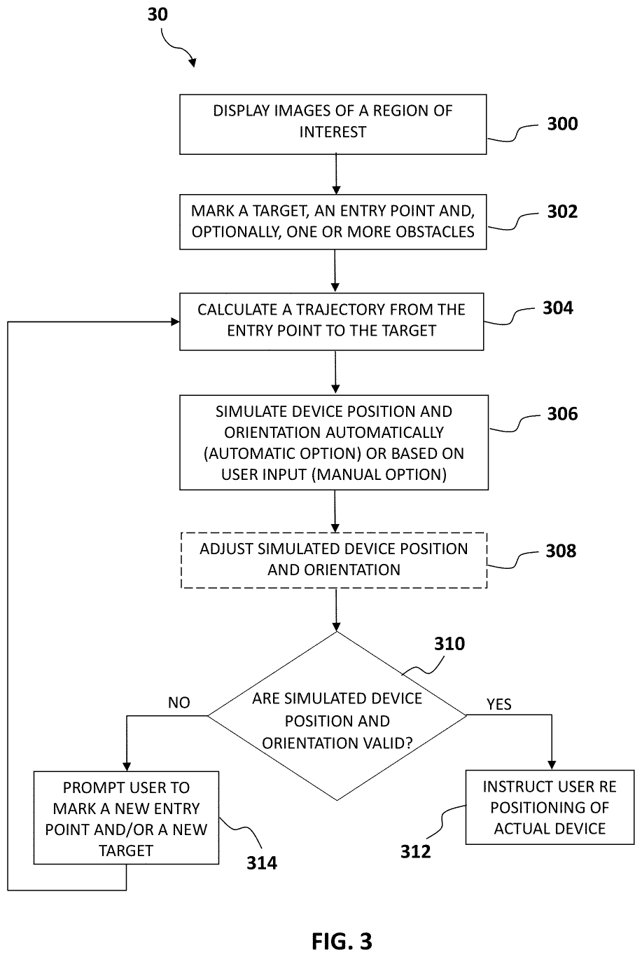

Reference is now made to FIG. 3, which illustrates a flowchart 30 showing the steps in an exemplary method for providing assistance to a user in positioning and orienting a medical device (e.g., automated insertion device) on, or in close proximity to, the subject's body, such that upon coupling a medical instrument (e.g., needle, probe) to the device, the instrument will be accurately positioned and oriented relative to the chosen entry point on the subject's skin, and will be able to follow the calculated trajectory until it reaches the target in the subject's body. According to some embodiments, one or more of the steps shown in FIG. 3 may be optional and one or more of the steps may be repeated.

At step 300, one or more images of the region of interest are displayed on a monitor. In some embodiments, the images are obtained by imaging initiated immediately prior to the initiation of the medical procedure. In other embodiments, the images are obtained from the medical records of the subject, such as images taken days, or even weeks, prior to the execution of the medical procedure. In such cases, the method shown in FIG. 3 may be carried out independently from the patient's presence, and be available to the user (e.g., physician) as a stand-alone software application.

At step 302, the target and the entry point are marked on the displayed image/s, for example, on one or more image-views generated from a set of images (or “slices”, or “image-frames”). Such image-views may be, for example, image-views pertaining to different planes or orientations (e.g., axial, sagittal, coronal, pseudo axial, pseudo sagittal, pseudo coronal, etc.) or additionally generated views (e.g., trajectory view, tool view, 3D view, etc.). In some embodiments, areas which should be avoided en route to the target, may also be marked on the image/s. Such areas may include, for example, bones, blood vessels, nerves, internal organs and/or implanted medical devices. In some embodiments, the user may mark the target and an entry point, and optionally also the obstacle/s, on the image/s. In other embodiments, the processor may be configured to automatically identify and mark at least one of the target, the optimal entry point and the obstacles, and the user may, optionally, be required to confirm or adjust the processor's proposed markings. In such embodiments, the target, obstacle/s, and optimal entry point may be identified/suggested using known image processing techniques based on the displayed images of the region of interest and/or on data obtained from previous similar procedures, using machine learning and/or deep learning algorithms.

At step 304, a trajectory from the entry point to the target is calculated. In case obstacles were marked on the initial image/s, the trajectory is calculated such that it avoids the obstacles. Although a linear trajectory is generally preferred, a linear trajectory may not always be possible to plan, due to the physical location of the target, the presence of obstacles, etc., thus the planned trajectory may be non-linear and have a certain degree of curvature. A maximal allowable curvature level may be determined, and it may depend, for example, on the type of instrument intended to be used in the procedure and its characteristics (e.g., diameter (gauge)). In some embodiments, a calculated trajectory is considered valid if its curvature does not exceed a predetermined threshold. In some embodiments, the trajectory is calculated based on the displayed images of the region of interest and the marked locations of the entry point, target and, optionally, obstacle/s. In some embodiments, the trajectory may be calculated based also on data obtained from previous similar procedures using machine learning and/or deep learning algorithms. In some embodiments, the calculated trajectory is a planar trajectory. In some embodiments, the calculated trajectory is a 3D trajectory. In some embodiments, the 3D trajectory may be calculated by determining a pathway on each of two two-dimensional (2D) planes and superpositioning the two planar trajectories to form a three-dimensional trajectory, as disclosed, for example, in abovementioned International Patent Application No. PCT/IL2020/051219. In some embodiments, the two planes are perpendicular to each other. In some embodiments, the planning of each of the two planar trajectories and the controlled steering of the instrument may be based on a model of the medical instrument as a flexible beam having a plurality of virtual springs connected laterally thereto to simulate lateral forces exerted by the tissue on the instrument, calculating the trajectory through the tissue on the basis of the influence of the plurality of virtual springs on the instrument, and utilizing an inverse kinematics solution applied to the virtual springs model to calculate the required motion to be imparted to the instrument to follow the planned trajectory, as described in abovementioned U.S. Pat. No. 8,348,861.

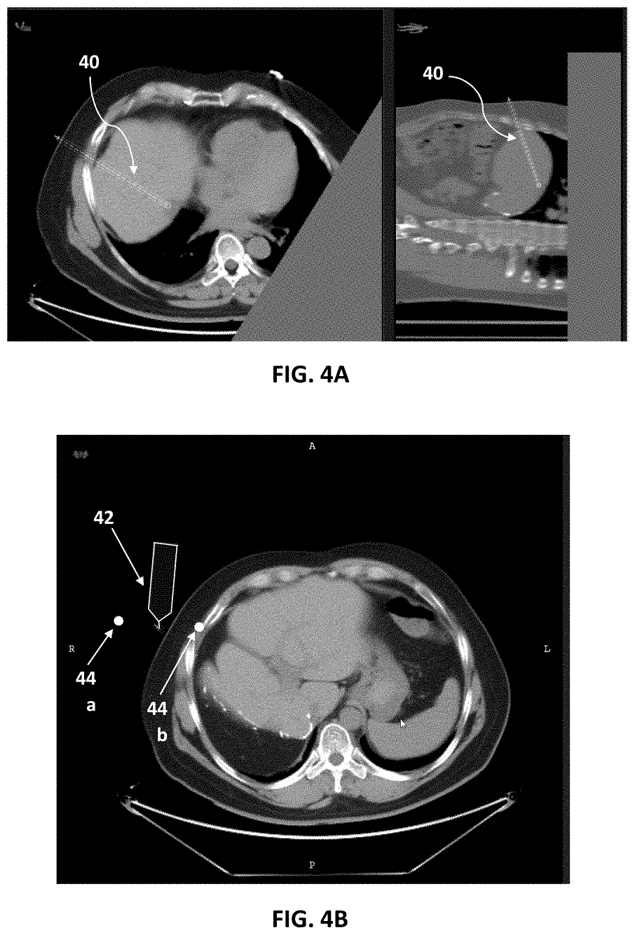

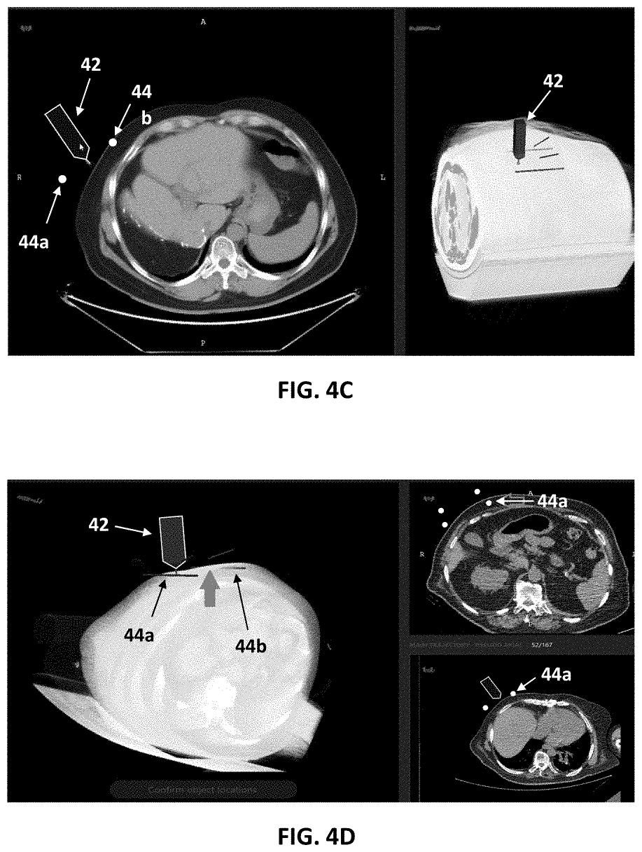

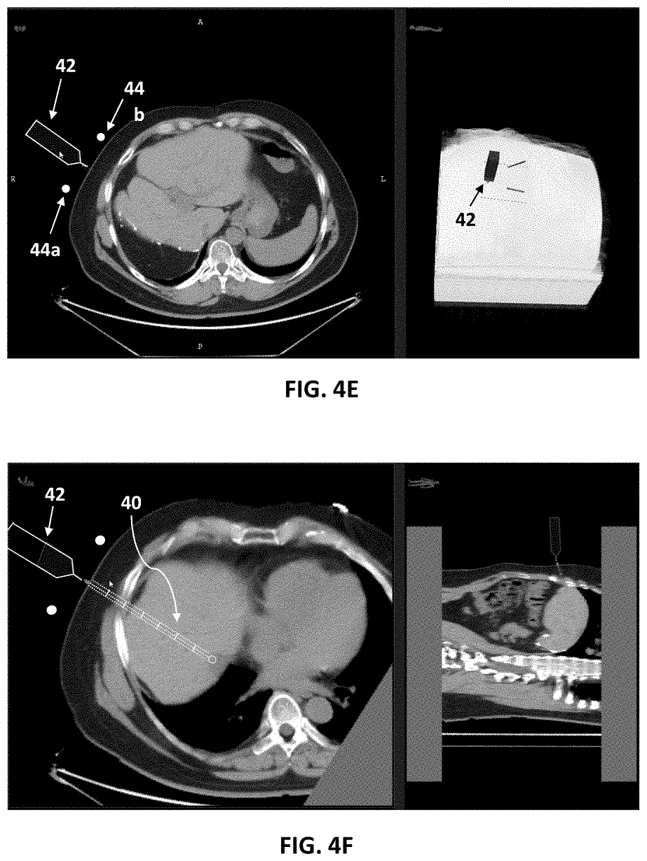

FIG. 4A shows a calculated trajectory 40 from an entry point to a target in the subject's body displayed on CT images, an axial plane view on the left-hand side and on a sagittal plane view on the right-hand side.

At step 306 of FIG. 3, the position and orientation of the medical device on the subject's body, or in proximity thereof, are simulated and displayed on the monitor using a virtual medical device. In some embodiments, the medical device is a robotic arm, and the simulation may be of the position and orientation of the robotic arm's proximal end, e.g., its end effector. In some embodiments, the medical device may be a body-mountable robotic device, and a virtual representation of the device's end effector and/or the device's base (or registration elements disposed thereon) may form the displayed virtual medical device. In some embodiments, the virtual device may be displayed on the image-view/s at an arbitrary position and the user can then move and/or rotate the virtual device and determine the simulated position and orientation of the medical device relative to the subject's body (“manual positioning option”). In some embodiments, the virtual device may be displayed on the image-view/s at a position and orientation which are based on the processor's calculations or “educated guess” (“automatic positioning option”). In some embodiments, the user may be provided a choice between the manual positioning option and the automatic positioning option.

In the automatic positioning option, the simulated position and orientation suggested by the processor may be based on the displayed images of the region of interest and the calculated trajectory and/or on data obtained from previous similar procedures, using machine learning and/or deep learning algorithms. In some embodiments, the position and orientation recommended by the processor may be based, on one or more of the following parameters: scanning/registration limitations (such as maximal orientation angles about one or more of the X, Y and Z axes); device workspace limitations (such as feasible end effector rotation angles); parameters relating to the patient's body, such as the body's shape and/or its contour, which may be detected automatically or manually marked, as described elsewhere herein, extension of the patient's body surface (obtained using external vision systems, for example); user configurable restrictions, such as: the device's facing direction, maximal instrument length above the patient's skin surface (so as not to compromise the accurate steering of the instrument, in case the steering algorithm is based on the instrument being positioned inside the tissue, as well as to prevent “wasted” instrument length), etc.; device setup considerations, such as avoiding positioning the device directly above the target, etc. In some embodiments, the recommendation for positioning the device may take into account the effect that attaching the device and/or a mounting unit, such as an attachment frame, to the patient has on the patient's body. For example, the attachment of the device and/or the mounting unit, may apply pressure onto the body such that the patient's body surface, tissue layers and internal organs, including the target, may shift, be pushed down and/or be “squeezed” closer together. In some embodiments, a machine learning module may include a learning model for the effect that attaching the device and/or an attachment frame to the patient has on the patient's body and output a recommendation accordingly. The different patients' body types, internal anatomy, etc. may be some of the parameters included in the learning model. In some embodiments, the recommended device positioning may include visible safety margin which are based on the expected “squeezing” effect of attaching the device and/or an attachment frame to the patient's body.

In the manual positioning option, according to some embodiments, the user's input may be provided by means of a “drag and drop” method, i.e., the user may move and/or rotate the virtual device displayed on the monitor using the user interface, such as a computer mouse, a joystick or his/her own fingers (in case the monitor is a touch-screen), and release his/her “grip” of the virtual device, once the desired position and orientation are achieved. In some embodiments, the user interface may include virtual buttons for moving the virtual device in each of the X, Y and Z axis, separately or simultaneously (for example, dx, dy and dz buttons), and/or rotating the virtual device about each of the X, Y and Z axis, separately or simultaneously.

In some embodiments, simulating the device's position and orientation may include adjusting the patient's pose, by rotating the image, to simulate different patient poses (e.g., prone, supine, decubitus etc.). In the automatic positioning option, the image may be automatically rotated, should the processor's calculations determine that a different patient pose would be preferable for the specific procedure. In the manual positioning option, the user may rotate the image and simulate different device positions on the rotated image/s.

FIG. 4B shows a robotic body-mountable device, as displayed by means of a virtual device (e.g., robot) on CT images. In FIG. 4B the virtual device is positioned in an arbitrary position on the image-view, prior to the user moving and rotating the virtual device, to simulate the actual device's position and orientation (manual positioning option). FIG. 4C shows a simulated position and orientation of the device displayed on CT images, either as determined automatically by the processor, in the automatic positioning option, or as determined based on the user's input, in the manual positioning option, e.g., after moving and rotating the virtual device displayed in FIG. 4B. In some embodiments, the virtual device may be represented by a virtual control head (end effector), such as virtual control head 42, and by one or more virtual registration elements, such as registration elements 44a and 44b, which simulate the actual registration elements positioned, for example, on the base of the device. The relative position between virtual control head 42 and virtual registration elements 44a and 44b advantageously corresponds to their relative position in the actual automated device.

At optional step 308 of FIG. 3, the simulated position and orientation of the medical device on the subject's body (or in proximity thereof) may be adjusted. In the automatic positioning option, the adjustment may be based on user input. In some embodiments, the user may decide to adjust the recommended simulated position and/or orientation if data which is unavailable to the processor should be considered, for example if the simulated position is such that should the device be positioned on the body accordingly, it might exert undesired pressure on internal organs (e.g., stomach) and/or cause undesired pain to the patient (e.g., if placed directly on a bone). Further, in some embodiments, the processor may not be able to take into consideration body parts and/or other external anatomical limitations which are outside the scanned area and may physically prevent placing the device as simulated (such as breasts of a female patient). It can be appreciated that in embodiments in which the automatic positioning option utilizes machine learning and/or deep learning algorithms, some of the above parameters may already be taken into consideration in the calculation of the recommended position of the device. In some embodiments, there may be additional patient specific considerations which may require adjustment of the automatically simulated device position, such as skin conditions (e.g., a rash, a bruise) and/or other patient specific sensitivities. In some embodiments, the user's adjustments may be provided by means of “drag and drop” and/or virtual buttons, as described above.

In some embodiments, in the manual positioning option or after the processor's simulated position and/or orientation of the device has been adjusted based on the user's input in the automatic positioning option in the automatic positioning option, fine-tuning by the system processor may be required. Since the processor can “see” all the obtained images (e.g., CT scans/slices) together, whereas the user (e.g., physician) can see on the monitor only one image at a time (or multiple images showing different views (e.g., different planes) of the same location), there may be parameters which only the processor can take into consideration, such as the skin surface/contour in the surrounding area which will impact the stability of the device. Further, the simulated position of the device may be such that a portion of the virtual device, such as part of its base, penetrates the patient's body, which can be identified only in certain image-views. FIG. 4D shows an example of a simulated device position in which one of the virtual registration elements, element 44b, is shown penetrating the patient's body on the 3D view (left-hand side) and on the axial view (top right-hand side), whereas in the pseudo-axial view (lower right-hand side), virtual element 44a appears to be positioned above the patient's skin surface. Since virtual registration element 44b corresponds to an actual registration element positioned, for example, on the automated device's base, the simulated device position cannot be implemented in actuality. In some embodiments, the system software may automatically detect and mark on the image/s the surface of the patient's body, such that the simulated position (in the automatic positioning option) and/or the fine-tuning of the simulated position (in the manual positioning option, or following user initiated adjustments in the automatic positioning option) avoids crossing the marked body surface. Alternatively, the patient's body surface may be manually marked on the image/s by the user, optionally prior to the initial simulation of the device's position and orientation. In some embodiments, the processor may determine that a slight adjustment of the device's position and/or orientation is required in order to enable or better suit the planned trajectory. In some embodiments, the processor's adjustments to the simulated position and orientation determined by the user may be limited to a maximal distance/angle from the simulated position and orientation which was determined manually by the user. FIG. 4E shows the virtual device's adjusted position and orientation, displayed on CT images.

At step 310 of FIG. 3, it is determined if the simulated position and orientation of the medical device is valid. In some embodiments, determining if the simulated position and orientation is valid includes determining if the simulated position and orientation ensure alignment of the tip of the instrument with the insertion point, at the desired insertion angle, such that by the instrument (e.g., the tip thereof) following the planned trajectory, it will reach the target (based on its position in the displayed image/s). In some embodiments, in order to determine if the simulated position and orientation of the medical device is valid, it may be required to verify that the trajectory, as calculated in step 304, is still valid, given the simulated position and orientation of the medical device. In some embodiments, the processor may check parameters pertaining to “robot feasibility”, for example that the angles required from the end effector are within the end effector's feasible rotation range. In some embodiments, the processor may verify that the simulated position of the device does not include any portion of the device, such as part of its base, penetrating the patient's body, in order to determine that the simulated position and orientation are valid. Additional parameters may be positioning limitations which relate to registration limits, such as maximal orientation angles of the medical device about one or more of the X, Y and Z axes, with the reference being the patient bed, for example. Additional parameters may be user configurable parameters, such as maximal distance of the device from the patient's skin surface, avoiding positioning of the robot directly above the target, etc. FIG. 4F shows the simulated position and orientation of the device together with the calculated trajectory, after both have been determined to be valid, displayed on CT images.

If it is determined that the simulated position and orientation is valid, then in step 312 of FIG. 3, the user is instructed as to how to physically position the medical device on the subject's body (or in close proximity thereto) based on the final simulated position and orientation of the medical device. In some embodiments, the instructions to the user may be displayed on the monitor and/or provided as audio instructions, e.g., via one or more speakers. In some embodiments, the instructions may include movement and/or rotation instructions, i.e., distance and/or angular values. The reference for the movement/rotation instructions may be an absolute reference, such as the patient bed, the imaging device, etc., or it may be the patient itself, e.g., the patient's body surface, a specific patient organ, etc. In some embodiments, the movement and/or rotation instructions may include elevating the medical device from the surface of the patient's body, in either a parallel or an angular manner. The elevation may be carried out using a dedicated mechanism, as will be described hereinbelow, or by means of cushions/pillows, which the user may be instructed to place at certain locations under the medical device or under the attachment frame. In some embodiments, the instructions may include the recommended patient pose (e.g., prone, supine, decubitus etc.), if different from the patient pose in the images displayed at step 300, and if the simulation of the device's position and orientation required changing the pose by rotating the image-view. In some embodiments, the instructions to the user may further include a recommended scanning range for the registration of the medical device to the image space.

In some embodiments, an auxiliary positioning mechanism may be used to assist the user in the positioning and/or orienting the medical device based on the provided positioning instructions. The auxiliary positioning mechanism may be part of, or provided with, the automated medical device or a mounting unit (e.g., attachment frame), which is configured to be attached to the patient's body and to which the medical device is then coupled, as will be described hereinbelow. In some embodiments, the auxiliary positioning mechanism may be a stand-alone device. In some embodiments, the auxiliary positioning mechanism/device may be configured for manual use by the user, i.e., the user may be required to move/adjust certain elements of the positioning device according to the provided positioning instructions. In some embodiments, the auxiliary positioning mechanism/device may be automatic, i.e., it may include an electronic or electromechanical mechanism, which can be controlled by the processor/controller, such that the provided positioning instructions may be executed automatically.

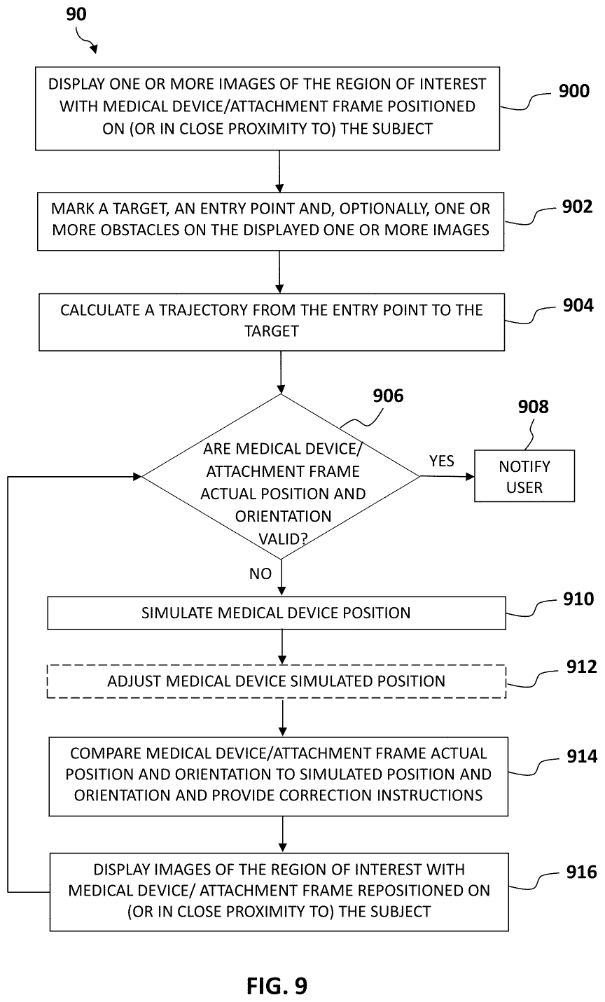

In some embodiments, after the medical device and/or the mounting unit (e.g., attachment frame) is positioned on (or in close proximity to) the subject's body based on the provided instructions, one or more images are obtained, to ensure that the medical device and/or the mounting unit is properly/accurately positioned and that its actual positioning is valid, as described, for example, in FIG. 9 hereinbelow.

In some embodiments, if it is determined that the simulated position and orientation is invalid, then at step 314, the user may be prompted to select a new entry point and/or adjust the marking of the target. In some embodiments, the processor may display on the image-view/s a recommended range (or area) on the patient's skin for selecting the new entry point. The recommended range may be based on the position of the target and/or on the simulated position and orientation of the device. Steps 304 to 310 are then repeated, until a valid simulated position is found. In some embodiments, if no valid simulated position and orientation are found, the processor may notify the user that the medical device cannot be utilized in the procedure at hand.

In some embodiments of the manual positioning option, if the simulated position and orientation are determined to be invalid, then before moving on to step 314, the processor may suggest to the user an alternative simulated position and orientation, which may be valid for the given calculated trajectory. In some embodiments of the automatic positioning option, if the simulated position and orientation is determined to be invalid, then before moving on to step 314, steps 308 and 310 may be repeated automatically by the processor (i.e., iterative calculations). In some embodiments, steps 308 and 310 may be repeated a limited number of iterations, for example up to 10 iterations, up to 5 iterations, up to 3 iterations, or any number of acceptable iterations, or for a limited period of time, for example up to 60 seconds, up to 45 seconds, up to 30 seconds, or any acceptable period of time. In some embodiments, if after the last iteration the simulated position and orientation is still determined to be invalid, then the algorithm moves on to step 314 described above. In some embodiments, each time step 308 is repeated, it is repeated relative to the current position and orientation of the virtual device, i.e., relative to the position and orientation of the virtual device as adjusted at the previous iteration of step 308. In other embodiments, each repetition of 308 step is executed relative to the original simulated position and orientation, i.e., once the simulated position and orientation is determined to be invalid, the position and orientation of the virtual device are returned to the initial position and orientation suggested by the processor (automatic positioning option) or determined by the user either with or without fine-tuning by the processor (manual positioning option).

Although the image-views shown in FIGS. 4A-4F were generated from CT scans, other imaging modalities, such as MRI and ultrasound, may alternatively be utilized. Further, it can be appreciated that although specific image-views are shown in FIGS. 4A-4F, different planes (e.g., axial, sagittal, coronal, pseudo axial, pseudo sagittal, pseudo coronal, etc.) or other views (e.g., trajectory view, tool view, 3D view) may be used in order to perform and/or display any of the above method steps.

In some embodiments, an auxiliary positioning mechanism may be used to assist the user in the positioning of the actual medical device based on the positioning instructions provided by the processor. The auxiliary positioning mechanism may be part of, or provided with, the automated medical device. In some embodiments, the auxiliary positioning mechanism may be a stand-alone device. In some embodiments, the auxiliary positioning device may be part of, or configured for coupling to, the mounting unit (e.g., attachment frame). In some embodiments, the auxiliary positioning device may be part of, or configured for coupling to, an aiming apparatus, which may be removably couplable to a mounting unit (e.g., attachment frame), as will be described hereinbelow. In some embodiments, the auxiliary positioning mechanism/device may be used manually by the user, i.e., the user may be required to manually move certain elements of the positioning device according to the provided positioning instructions. In some embodiments, the auxiliary positioning mechanism/device may be an automatic mechanism/device, i.e., it may include an electromechanical mechanism controllable by the processor/controller, such that the positioning instructions can be automatically executed, immediately following the generating of the instructions, or after obtaining user confirmation to execute the instructions. In some embodiments, the mechanical, electrical and/or electromechanical mechanism may be able to apply accurate movements and/or rotations to the medical device and/or the attachment frame. In some embodiments, the mechanical, electrical and/or electromechanical mechanism may be able to apply accurate movements and/or rotations to one or more physical elements which represent/simulate one or more elements of the medical device, and the user is then required to apply these movements and/or rotations to the medical device itself. In some embodiments, the auxiliary positioning mechanism/device may be configured to elevate the medical device from the patient's body, either automatically or via manual manipulation by the user. The elevation may be parallel to the surface of the patient's body and/or of the attachment frame, or it may be at an angle relative to the surface of the patient's body and/or of the attachment frame, as will be described hereinbelow.

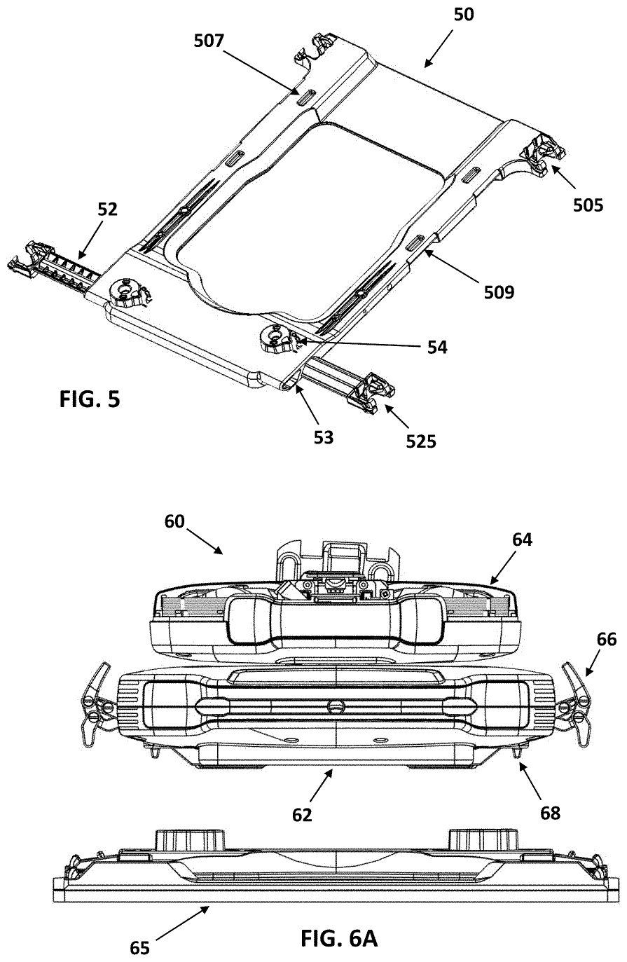

In some embodiments, the medical device (e.g., the insertion and steering device 2 shown in FIG. 2A hereinabove) is body-mountable. In some embodiments, the medical device may be attached to the subject's body directly, e.g., using one or more straps. In other embodiments, the medical device may be attached to the subject's body using a mounting unit, such as an attachment frame, which is configured to be secured to the subject's body using one or more straps, for example, and to receive the medical device thereon. FIG. 5 shows an exemplary attachment apparatus configured as a frame surrounding an opening which allows access of the medical instrument to the subject's body upon securing the medical device to the attachment frame and coupling the medical instrument to the medical device.

As shown in FIG. 5, the attachment frame 50 may include one or more extending members (“cranes”) 52 to which one or more straps (not shown) may be coupled. The cranes may extend (or be extended) away from the attachment frame 50, allowing the straps to pull the frame 50 in a direction substantially vertical to the plane of the frame 50, thus maximizing the mounting force while minimizing the contact area of the straps with the patient's skin. In some embodiments, the cranes 52 may be separate units removably couplable to the attachment frame 50. In other embodiments, the cranes 52 may be integral with the attachment frame 50. The cranes 52 may be disposed at (or couplable to) both ends of the attachment frame 50, or they may be disposed only at one end of the frame 50, as shown in FIG. 5. In order to accommodate different body types having different circumferences, as well as different locations on the subject's body having different circumferences, the cranes 52 may have a fixed length, and the attachment frame 50 may be provided with a plurality of cranes 52 having varying lengths, which the user can choose from. Alternatively, at least one of the cranes 52 may be configured as a lengthening member, e.g., configured as telescopic members. In some embodiments, as shown in FIG. 5, the cranes 52 may be deployable from within dedicated channels 53 formed in the frame 50. The frame 50 may include a separate channel 53 for each of the cranes 52 or a single channel may accommodate a set of cranes, for example, the two cranes which extend away from the same end of the frame 50 in opposite directions from each other. In some embodiments, each crane 52 may be associated with a locking knob 54, which is used for locking the position of the crane 52 within its corresponding channel 53, once it is moved out of (or into) the channel to its desired position, i.e., its desired length extending outwardly from the attachment frame 50. The movement of the cranes in and out of the channel may be automatic or manual. Using an attachment frame 50 with deployable cranes 52 may minimize the total strapping loads on the subject, by maximizing the mounting area and minimizing the strapping force losses, as described, for example, in co-owned International Patent Application Publication No. WO 2019/234,748, to Galili et al, which is incorporated herein by reference in its entirety.

In some embodiments, the distal end of the crane 52, i.e., the end which is farthest from the frame 50 when the crane 52 is deployed, may comprise a crane connector 525 to which a strap can be coupled to secure the attachment frame 50 to the patient's body. In case only one end of the frame 50 is provided with one or more cranes 52, as shown in FIG. 5, for example, the opposite end of the attachment frame 50 may include frame connectors 505, to which additional straps can be coupled. It can be appreciated, that the crane connectors 525 and the frame connectors 505 may be either similar connectors or different types of connectors. Further, the two opposing crane connectors 525 may be either similar or they may differ from each other, and the two opposing frame connectors 505 may be either similar or they may differ from each other.

The attachment frame 50 may further include one or more grooves 507, which are sized and shaped to receive corresponding protrusions (not shown in FIG. 5) located at the bottom of the medical device, to facilitate and ensure alignment between the medical device and the attachment frame 50 upon positioning the medical device on the attachment frame 50. The grooves 507, and hence the corresponding protrusions, may be distributed in an asymmetrical manner, to better ensure proper alignment between the medical device and the attachment frame 50 and prevent unintentional reverse placement of the medical device on the frame 50. In some embodiments, the bottom of the medical device may include the grooves and the attachment frame may include the corresponding protrusions.

The attachment frame 50 may further include one or more notches 509 for receiving and engaging with corresponding one or more latches (not shown in FIG. 5) of the medical device, to secure the medical device to the attachment frame 50 after it has been properly positioned thereon.

In some embodiments, additional elements (e.g., stoppers (not shown)) may be used to ensure proper positioning of the medical device on the attachment frame 50, as well as to prevent movement of the medical device relative to the attachment frame 50 after it has been positioned on the frame 50 and before is has been secured thereto.

In some embodiments, the utilized mounting unit (e.g., attachment frame) may be configured to allow adjusting the position and orientation of the medical device in the plane of the attachment frame, or in a plane parallel to the plane of the attachment frame, as disclosed, for example in co-owned U.S. Patent Application Publication No. 2019/125,397, to Arnold et al, which is incorporated herein by reference in its entirety. In some embodiments, the attachment frame may include a stationary plate and a movable plate, which is coupled to the stationary plate such that it can be moved relative to the stationary plate. In some embodiments, the attachment frame may include also a rotating plate coupled to the movable plate, to enable rotation of the medical device about a vertical axis (yaw). The rotation range may be 360 degrees, or it may be otherwise restricted. In some embodiments, the movement of the movable plate and/or the rotation of the rotatable plate may be executed manually by the user or automatically by the processor/controller.

In some embodiments, the attachment frame may include a parallel lifting member (or mechanism), or an angular lifting member (or mechanism), as disclosed, for example, in abovementioned International Patent Application Publication No. WO 2019/234,748. The lifting member may include at least one rail and at least one fixator configured to fixate the position of the medical device along the at least one rail. The lifting member may be removably couplable to the attachment frame, rigidly coupled to the attachment frame or an integral part of the attachment frame. Such lifting members may enable elevating and/or tilting the medical device relative to the surface of the patient's body and/or relative to the surface of the attachment frame.

In some embodiments, one or more registration elements (not shown) may be positioned on the attachment frame 50, for determining the attachment frame's and thus (the medical device's) position and/or orientation in the image space during image-guided procedures, for example, as disclosed in co-owned U.S. Pat. No. 10,806,523, to Roth et al, which is incorporated herein by reference in its entirety.

In some embodiments, the attachment frame 50 may be reusable, such that the same frame can be used repeatedly in several medical procedures performed on different patients. In other embodiments, the attachment frame 50 may be disposable, such that a new frame is used in each medical procedure, and the frame is disposed of after completing the procedure.

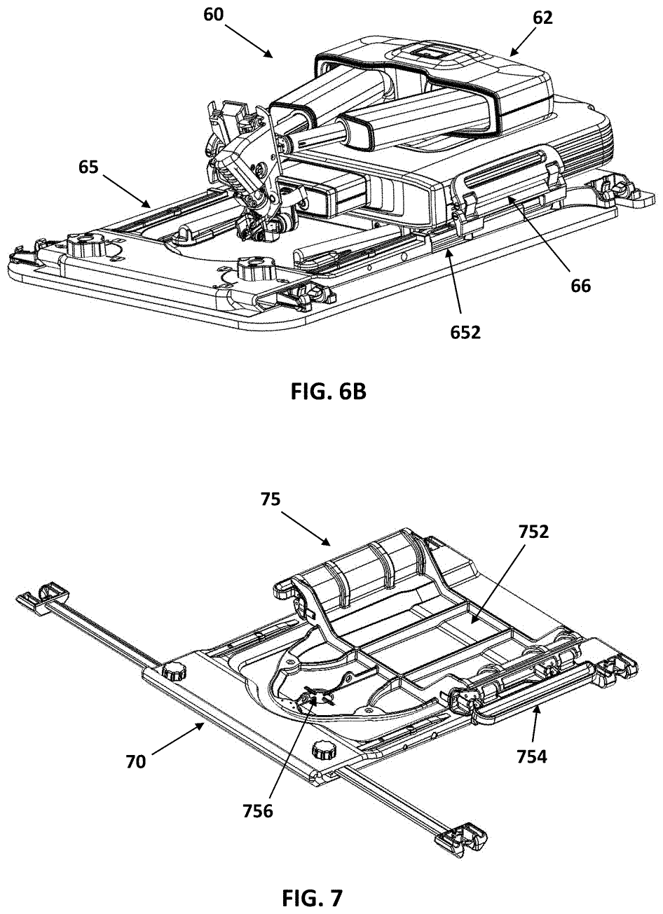

FIG. 6A shows a rear view of an exemplary medical device 60 and an exemplary attachment frame 65, prior to positioning of the medical device 60 on the attachment frame 65. In some embodiments, the medical device housing 64 may include latches, such as latches 66, disposed on opposite sides of the housing 64, to couple the device 60 to the attachment frame 65. FIG. 6A shows the latches 66 in an open state. The latches 66 may be an integral part of the device housing 64, or they may be separate units rigidly connected to the housing 64. The device housing 64 and/or the latches 66 may have protrusions 68, which are sized and shaped to be received within corresponding grooves located on the upper surface of the attachment frame, as discussed above in FIG. 5, for example, to facilitate and ensure proper alignment between the medical device 60 and the attachment frame 65 upon coupling. In some embodiments, the coupling of the medical device to the attachment frame may be facilitated by other means, such as by a magnetic connection, or any other appropriate coupling mechanism.

In some embodiments, the medical device 60 is reusable, at least in part. In such embodiments, the medical device 60 may be positioned on the attachment frame 65 only after the medical device 60 has been covered with a sterile drape (not shown).

FIG. 6B shows the medical device 60 positioned on the attachment frame 65, before closing of the latches 66 using the corresponding notches 652 of the attachment frame 65. In some embodiments, the latches 66 may include a spring (not shown) which maintains them in an open position when they are not grasped by the notches 652 of the frame 65. In some embodiments, the medical device 60 (e.g., its latches 66) and/or the attachment frame 65 may include at least one visual or auditory indicator (not shown), to indicate to the user that the medical device 60 is properly positioned on and/or properly secured to the attachment frame 65. The indicator may be mechanically and/or electronically activated.

FIG. 7 shows an exemplary aiming apparatus (also referred to as “aiming jig” or “alignment apparatus”) 75 coupled to an attachment frame 70. In some embodiments, the medical device which is to be coupled to the attachment frame 70 is a device for inserting a medical instrument, such as a needle or an introducer, into the subject's body, in order to perform a biopsy, deliver fluid to a target within the body, perform ablation, etc. Prior to inserting the medical instrument into the subject's body, whether the insertion is done manually by the physician or automatically by the insertion device, the physician typically marks the entry point on the subject's body. Therefore, the attachment frame 70 should be attached to the subject's body such that once the insertion device is coupled to the attachment frame 70, the tip of the medical instrument is located directly above the entry point, or can be easily aligned with the entry point. In some embodiments, an aiming apparatus 75 may be coupled to the attachment frame 70 to facilitate the proper positioning of the attachment frame 70 relative to the marked entry point. Once the proper positioning is achieved, the physician can remove the aiming jig 75 and couple the insertion device to the attachment frame 70.

As shown in FIG. 7, the aiming apparatus 75 may comprise a plate 752 which, in some embodiments, may be couplable to the attachment frame 70 in a manner similar to that by which the medical device is coupled to the attachment frame 70, for example, as shown in FIGS. 6A-6B above. In some embodiments, the aiming apparatus 75 may include latches 754 which engage with notches (not shown) of the attachment frame 70, to secure the connection of the aiming apparatus 75 to the attachment frame 70. The aiming apparatus 75 may further include an opening 756 in its base 752, which is located at a location corresponding to the expected location of the tip of the medical instrument relative to the attachment frame 70 when the insertion device is coupled to the attachment frame 70. The opening 756 may be provided with a cross, or any other mark, to further point to the expected location of the medical instrument's tip. Thus, the user should place the attachment frame 70 with the aiming apparatus 75 coupled thereto on the subject's body, and secure the attachment frame 70 to the body using the straps, for example, while maintaining the opening 756 of the aiming apparatus 75 aligned with the entry point marked on the subject's body. Once the attachment frame 70 is securely attached to the subject's body, the user can open the latches 754 of the aiming apparatus 75, or any alternative coupling mechanism which may be used, remove the aiming apparatus 75 from the attachment frame 70 and then couple the insertion device to the attachment frame 70.

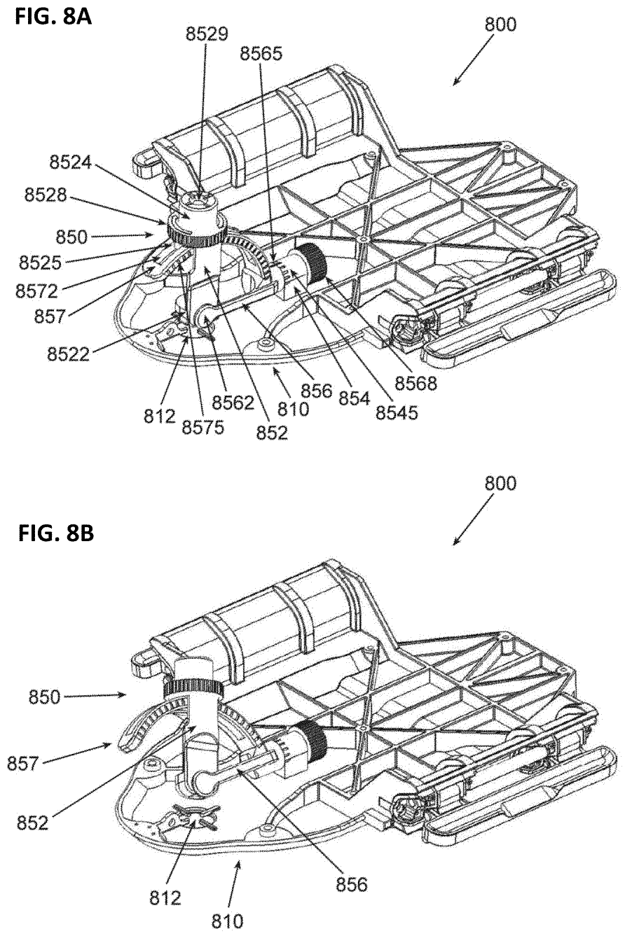

FIGS. 8A-8B show an exemplary aiming apparatus which includes an auxiliary positioning mechanism. The shown aiming apparatus may be used not only for ensuring alignment of the tip of the medical instrument with the marked entry point, but also for assisting the user in positioning the attachment frame, and thus the medical device, according to the simulated position and orientation determined to be valid by the processor, for example, in step 310 of the method shown in FIG. 3.

FIG. 8A shows the exemplary aiming apparatus 800 comprising an auxiliary mechanism, for example orienting mechanism 850, which may be either removably couplable to the base 810 of the aiming apparatus 800 or an integral part thereof In some embodiments, the aiming apparatus may be configured to be moved in a parallel manner, relative to the attachment frame, in front-back and/or left-right directions, e.g., by virtue of rails formed in the attachment frame, to assist the user in positioning the medical device. In some embodiments, the rails formed in the attachment frame may further enable moving the medical device in a parallel manner relative to the attachment frame. In some embodiments, the aiming apparatus may include a parallel lifting member (or mechanism), or an angular lifting member (or mechanism), as disclosed, for example, in abovementioned International Patent Application Publication No. WO 2019/234,748. Such lifting members may further assist the user in positioning the medical device when elevating and/or tilting the medical device relative to the surface of the patient's body and/or relative to the surface of the attachment frame is required. In some embodiments, the orienting mechanism 850 may include an orienting member 852, which may simulate the medical instrument or the end effector of the insertion device, such as the insertion device shown in FIG. 2A. In some embodiments, the orienting mechanism 850 may further include an anchoring base 854, which is coupled, either rigidly or removably, to the base 810. In some embodiments, a rotating arm 856 may be coupled to the stationary anchoring base 854, such that the rotating arm 856 can rotate about its axis. The rotating arm 856 may include, or be coupled to, an upper arch 857 having an elongated groove 8572 extending along at least a portion of the arch's length. The upper arch 857 may be configured to rotate together with the rotating arm 856. In some embodiments, the lower portion 8522 of the orienting member 852 may be coupled to the distal end 8562 of the rotating arm 856 by means of a hinge, located above an in juxtaposition to the opening 812 of the aiming apparatus' base 810, and the top portion 8524 of the orienting member 852 may be coupled to the arch 857 such that a protrusion 8525 of the top portion 8524 is positioned within the groove 8572. In such embodiments, the orientation of the orienting member 852 may be adjusted by rotating the rotating arm 856 about its axis (simulating the end effector' s left-right rotation capabilities), for example using a knob 8568, or by pivoting the orienting member 852 about the axis of the hinge with the orienting member's protrusion 8525 moving within the arch's elongated groove 8572 (simulating the end effector's front-back rotation capabilities), or a combination thereof (simulating the end effector's entire rotation range). In some embodiments, the orienting member 852 may be configured as a hollow member, the cross-section of which may be circular, oval, rectangular, or any other suitable cross-section. In some embodiments, the orienting member 852 may be configured as a cylinder, for example, with a thin channel formed along the length of the orienting member 852, between its top portion 8524 and its lower portion 8522, to allow viewing of the entry point, or access to the entry point, e.g., using a thin rod, a laser beam, etc., from the top portion 8524 of the orienting member 852, through the length of the orienting member 852 and through the opening 812 of the aiming apparatus' plate 810. In other embodiments, and as shown in FIGS. 8A-8B, the top portion 8524 and lower portion 8522 of the orienting member 852 may be connected by a single wall, with the top portion 8524 having an opening 8529 and the lower portion having an opening (not shown) aligned with the opening 8529 of the top portion 8524, to allow viewing of the entry point, or access to the entry point, e.g., using a thin rod, a laser beam, etc., from the top portion 8524 of the orienting member 852, through the length of the orienting member 852 and through the opening 812 of the aiming apparatus' plate 810. It can be appreciated, that the top portion 8524 and lower portion 8522 of the orienting member 852 may be connected by more than one wall, for example two walls, three walls, etc. In some embodiments, the thin rod, or any other elongated member which can be inserted through the top portion 8524 of the orienting member 852 until it reaches the entry point marked on the patient's body, may include marks (a scale), such that when the rod reaches the entry point, the user can measure the distance from the entry point to the top portion 8524 of the orienting member 852, and translate that to the length of the medical tool which will extend from the entry point to the top end of the insertion module disclosed in abovementioned U.S. Patent Application Publication No. 2017/258,489, for example, should the end effector be positioned and oriented as the aiming apparatus' orienting member 852 is positioned and oriented. Such measurement will enable the user to determine if the specific position and orientation will allow the instrument, having a fixed length, to reach the target, given its depth within the body.

In some embodiments, the orienting mechanism 850 may be configured such that the rotation angles of the rotating arm 856 and of the orienting member 852 relative to the rotating arm 856, are limited to the maximal angles which can be reached by the insertion device, e.g., by the end effector of the insertion device. Thus, in case an attempt to align the attachment frame, using the aiming apparatus 800, according to the desired insertion angle, fails, the user can adjust the position and/or the angle of the attachment frame relative to the patient's body, e.g., using cushion/s. In some embodiments, the attachment frame may include a parallel lifting member (or mechanism), or an angular lifting member (or mechanism), as disclosed, for example, in abovementioned International Patent Application Publication No. WO 2019/234,748. Such lifting members may enable elevating and/or tilting the medical device coupled thereto relative to the patient's body surface, either manually or automatically.

In some embodiments, the anchoring base 854 may be provided with notches/marks 8545, and the proximal end of the rotating arm 856 may be provided with an indicator 8565, which is positioned adjacent the notches 8545, to indicate to the user, and assist him/her in controlling, the degree of rotation of the rotating arm 856. In some embodiments, the arch 857 may be provided with notches/marks 8575 disposed along at least a portion of the arch 857, to indicate to the user, and assist him/her in controlling, the degree of pivoting of the orienting member 852 relative to the rotating arm 856.

In some embodiments, once the desired orientation of the orienting member 852 has been reached, the user can lock the orienting member 852 at the desired orientation, using one or more locking mechanisms. In some embodiments, the rotating arm 856 may comprise a knob 8568, which can be rotated, for example clockwise, to lock the orientation of the rotating arm 856 via friction with the anchoring base 854, and prevent further rotation of the rotating arm 856 about its axis. Similarly, in some embodiments, the orienting member 852 may comprise a knob 8528, which can be rotated, for example clockwise, to lock the orientation of the orienting member 852 relative to the rotating arm 856, i.e., by locking the position of the protrusion 8525 within the elongated groove 8572, via friction with the arch 857, and prevent further movement of the orienting member 852 along the arch 857.

FIG. 8B shows the aiming apparatus 800 after the orienting member 852 has been set in the desired orientation relative to the opening 812 in the plate 810. In some embodiments, setting the orienting member 852 to the desired angle relative to the opening allows the user to verify, prior to coupling the insertion device to the attachment frame, that the insertion angle required in order for the medical instrument to follow the planned trajectory from the marked entry point to the marked target, is achievable by the device (specifically, by its end effector). In some embodiments, the orienting member 852 may assist the user in adjusting the orientation of the insertion device. Such adjustment may be required if the required insertion angle is determined not to be achievable by the insertion device and/or if the required patient pose (e.g., decubitus) does not enable positioning the attachment frame parallel to the body surface. Adjustment of the orientation of the medical device relative to the patient's body surface may be achieved by adjusting the orientation of the attachment frame relative to the patient's body surface and/or by activating an angular lifting mechanism of the attachment frame, in case the attachment frame comprises such a mechanism. In some embodiments, the user may set the orienting member 852 in an orientation opposite to the orientation which aligns the orienting member with the opening 812 at the desired angle, i.e., if the required orientation is such that the orienting member 852 is to be rotated backward and to the right, as shown in FIG. 8B, the opposite orientation is set by rotating the orienting member 852 forward and to the left, by the same angles relative to the orienting member's “zero” position shown in FIG. 8A. Once the orienting member 852 is set at the opposite orientation, the orientation of the attachment frame and/or of an angular lifting mechanism of the attachment member may be adjusted (up-down (pitch) and/or left-right (roll)) until the orienting member is positioned vertically and directly above the entry point, such that the entry point can be seen through the a thin channel formed along the length of the orienting member 852. This orientation of the medical device will allow inserting the medical instrument from the entry point to the target point with the end effector oriented at its “zero” position, i.e., substantially perpendicular to the base of the insertion device.

In some embodiments, after the attachment frame has been secured to the patient's body in alignment with the entry point, and while the aiming apparatus 800 is still coupled to the attachment frame, imaging of the region of interest, which includes both the entry point and the target, may be initiated, to verify, prior to removal of the aiming apparatus 800 from the attachment frame and coupling of the insertion device thereto, that the set entry angle matches the desired entry angle according to the planned trajectory.

In some embodiments, the orienting member 852 may include one or more registration elements, e.g., fiducial markers, such that the position of the orienting member 852 relative to the image space can be determined. In some embodiments, the determined position of the orienting member 852 relative to the image space can then be used, for example by the insertion system's software, in order to automatically position the medical instrument, or the insertion device's end effector to which the medical instrument is coupled, in the desired position and orientation for the commencement of the medical procedure.

In some embodiments, the attachment frame may include a plurality of visible markings, e.g., notches, grid lines, to assist the user if repositioning of the attachment frame is required. The markings may be configured as registration (e.g., fiducial) elements, such that they can be detected by an imaging system, e.g., in a CT image. In some embodiments, the markings may be detectable by an external vision apparatus, such as a camera, which may be used during the positioning process, such that data obtained from the camera can be integrated into the positioning algorithm.

It can be appreciated, that alternative positioning and/or orienting mechanisms may be used. For example, in some embodiments, a ball and socket mechanism (not shown) may be utilized, e.g., a ball having a hollow channel formed therethrough (a bead-like configuration) may be coupled to the opening 812 of the aiming apparatus' plate 810, such that it can be rotated therein and locked at the desired orientation.

Reference is now made to FIG. 9, which illustrates a flowchart 90 showing the steps in an exemplary method for assisting the user in positioning and/or orienting a medical device. According to some embodiments, one or more of the steps shown in FIG. 9 may be optional and one or more of the steps may be repeated.

At step 900, one or more images of the region of interest are displayed, after the user has positioned the medical device and/or the attachment frame on the subject, or in close proximity thereto, based on his/her knowledge and prior experience. In case an attachment frame is utilized, the one or more images may be obtained after the attachment frame has been positioned on the subject's body, but before coupling the medical device to the frame. Further, the one or more images may be obtained after an aiming jig, such as the aiming jig described in FIGS. 8A-8B, has been coupled to the attachment frame, and, optionally, after an auxiliary positioning mechanism (or device), for example the orienting member of the aiming jig, has been positioned and rotated to the orientation which the physician estimates, based on his/her knowledge and prior experience, will align the tip of the medical instrument with the insertion point at the insertion angle which will enable the instrument to be steered by the medical device until it reaches the target.

At step 902, the target and the entry point are marked on the displayed image/s. In some embodiments, areas which should be avoided en route to the target, such as bones, blood vessels, nerves, etc., may also be marked on the image/s. In some embodiments, the marking is done by the user. In such embodiments, a feasible (achievable) range for the location of an entry point on the patient's skin, for the given target position and/or the given position of the medical device/attachment frame, may be indicated by the processor, e.g., marked on the image-view/s. In other embodiments, the processor may be configured to identify and mark at least one of the target, the entry point and the obstacle/s, and the user may, optionally, be required to confirm and/or adjust the processor's proposed markings. In such embodiments, the target (and obstacle/s) may be identified using known image processing techniques, and an optimal entry point may be suggested based on the obtained images of the region of interest and/or data obtained from previous similar procedures using machine learning and/or deep learning capabilities.

At step 904, a trajectory from the entry point to the target is calculated. In case obstacles were marked on the initial image/s, the trajectory is calculated such that it avoids the obstacles. Although a linear trajectory is generally preferred, a linear trajectory may not always be possible to plan, due to the physical location of the target, the presence of obstacles, etc., thus the planned trajectory may have a certain degree of curvature. A maximal allowable curvature level may be determined, and it may depend on the type of instrument intended to be used in the procedure and its characteristics (e.g., diameter (gauge)). In some embodiments, a calculated trajectory is valid if its curvature does not exceed a predetermined threshold. In some embodiments, the trajectory is calculated based on the displayed images of the region of interest and the marked locations of the entry point, target and obstacle/s. In some embodiments, the trajectory may be calculated based also on data obtained from previous similar procedures using machine learning and/or deep learning capabilities.

At step 906, it is determined if the current (actual) position and orientation of the medical device and/or of the attachment frame are valid, i.e., if the current position and orientation of the device and, optionally, also of the auxiliary positioning mechanism/device, as set by the user prior to the execution of step 900, ensure alignment of the tip of the instrument with the insertion point, at the desired insertion angle, such that by the tip following the planned trajectory, it will reach the target (based on the position of the target in the obtained image/s). In some embodiments, in order to determine if the simulated position and orientation of the medical device is valid or not, it may be required to verify that the trajectory, as calculated in step 904, is still valid. In some embodiments, parameters pertaining to “robot feasibility” may be checked at step 906, for example that the angles required from the end effector are within the end effector's feasible rotation range, etc. Additional parameters may be positioning limitations which relate to scanning/registration limits, such as maximal orientation angles of the medical device about one or more of the X, Y and Z axes. Additional parameters may be user configurable parameters, such as maximal distance of the device from the patient's skin surface, avoiding positioning of the robot directly above the target, etc.

If it is determined that the current position and orientation of the medical device or of the attachment frame (and thus of the device which is to be coupled thereto at the frame's current position) is valid, then at step 908, the user is notified. In some embodiments, the notification may be in an active form, i.e., a notification such as “Position Valid” may be displayed on the monitor and/or a similar audio notification may be provided via speakers. In some embodiments, the notification may be in a passive form, i.e., the procedure flow will automatically continue to the next step (e.g., if the medical device is attached to the patient's body, either directly or using an attachment frame, the user may be instructed to steer the instrument tip to the entry point), indicating that the current position and orientation of the device (or the attachment frame) has been determined as valid.

If it is determined that the current position and orientation of the device (or the attachment frame) is invalid, then at step 910, a new position and orientation of the medical device on the subject's body (or in proximity thereof) are simulated and displayed on the monitor using a virtual device. In some embodiments, the medical device is a robotic arm, and the simulation may be of the position and orientation of the robotic arm's proximal end, e.g., its end effector. In some embodiments, the medical device may be a body-mountable robotic device, and the simulation may be of the device's end effector and/or the device's base, which is intended for positioning on the subject's body, either directly or by means of an intermediary element, such as the attachment frame of FIG. 5. In some embodiments, the virtual device may be displayed on the image-view at an arbitrary position and the user can then move and/or rotate the virtual device and determine the simulated position and orientation of the medical device relative to the subject's body (“manual positioning option”). In some embodiments, the virtual device may be displayed on the image-view at a position and orientation which are based on the processor's calculations or “educated guess” (“automatic positioning option”). The processor's recommendation may be based on the displayed images of the region of interest and the calculated trajectory and/or on data obtained from previous similar procedures, using machine learning and/or deep learning capabilities. In some embodiments, the user can choose between the manual positioning option and the automatic positioning option. In some embodiments, the simulated/recommended position and orientation, in the automatic positioning option, may be based, inter alia, on scanning/registration limitations (e.g., to prevent imaging artifacts), parameters relating to the patient's body (e.g., body shape, etc.), user configurable restrictions (e.g., device facing direction), device setup considerations (e.g., avoiding target being directly under the device), the “squeezing” effect that attaching the device and/or the attachment frame has on the patient's body, etc., all as described hereinabove in relation to step 306 of FIG. 3. In the manual positioning option, according to some embodiments, the user's input may be provided by means of “drag and drop”, i.e., the user may move and/or rotate the virtual device displayed on the monitor using the user interface, such as a computer mouse, a joystick or his/her own fingers (in case the monitor is a touch-screen), and release his/her “grip” of the virtual device, once the desired position and orientation are achieved. In some embodiments, the user interface may include virtual buttons for moving the virtual device in each of the X, Y and Z axis, separately or simultaneously (for example, dx, dy and dz buttons), and/or rotating the virtual device about each of the X, Y and Z axis, separately or simultaneously. In some embodiments, simulating the device's position and orientation may include adjusting the patient's pose, by rotating the image, to simulate different patient poses, such as prone, supine, decubitus etc. In the automatic positioning option, the image may be automatically rotated, should the processor's calculations determine that a different patient pose would be preferable for the specific procedure. In the manual positioning option, the user may rotate the image and simulate different device positions on the rotated image/s.

At optional step 912, if required, the simulated position and orientation of the medical device on the subject's body (or in proximity thereof) are adjusted. In the automatic positioning option, the adjustment may be based on user input, which may be provided by means of “drag and drop” and/or virtual buttons, as described hereinabove. In some embodiments, the adjustments may be done automatically by the processor.

Automatic adjustment (e.g., for fine-tuning) may be required in the manual positioning option, after the user has determined the simulated position and orientation of the device and/or in the automatic positioning option, if after the processor has determined the simulated position and/or orientation of the device, the simulated position and orientation was adjusted by the user, as described hereinabove.

At step 914, the actual position and orientation of the medical device and/or of the attachment frame is compared to the (final) simulated position and orientation. In some embodiments, prior to comparing the actual position and orientation of the medical device and/or attachment frame to the simulated position and orientation of the medical device, the validity of the simulated position and orientation are determined, such that the actual position and orientation of the medical device and/or attachment frame is compared to the simulated position and orientation only if the simulated position and orientation are determined to be valid. In some embodiments, if the simulated position and orientation are determined to be invalid, a new simulated position and orientation of the device is generated, and then the validity of the new simulated position and orientation is determined prior to comparing the actual position and orientation to the new simulated position and orientation. In some embodiments, the calculation of new simulated position and orientation may be limited to a set time period and/or to a predetermined number of iterations. In some embodiments, if after the last iteration the simulated position and orientation is still determined to be invalid, the user may be prompted to select a new entry point and/or adjust the marking of the target.

In some embodiments, if the actual position and orientation deviate from the simulated position and orientation, instructions as to how to correct the current positioning so that it matches the simulated positioning, are provided. In some embodiments, the correction instructions may relate to the position and/or orientation of the device (or attachment frame) itself, i.e., how (which direction) and how much (distance/angles) to move and/or rotate the device. In some embodiments, the correction instructions may relate to the position and/or orientation of the end effector of the device. In some embodiments, the correction instructions may be a combination of instructions relating to the position and/or orientation of the device itself and instructions relating to the position and/or orientation of the end effector of the device. The correction instructions may be given as corrections relative to the current position and orientation or as absolute desired positioning relative to a known reference, e.g., the patient bed or the imaging device. In some embodiments, the correction instructions may be a combination of instructions relating to the position and/or orientation of the device itself and instructions relating to the position and/or orientation of the end effector of the device.

In some embodiments, an auxiliary positioning mechanism may be used to assist the user in applying the correction instructions provided by the processor to the position and/or orientation of the medical device. The auxiliary positioning mechanism may be part of, or provided with, the automated medical device or the attachment frame. In some embodiments, the auxiliary positioning mechanism may be a stand-alone device. In some embodiments, the auxiliary positioning mechanism/device may be configured for manual use by the user, i.e., the user may be required to move/adjust certain elements of the auxiliary positioning device according to the provided correction instructions. In some embodiments, the auxiliary positioning mechanism/device may be automatic, i.e., it may be controllable by the processor/controller, such that the provided correction instructions may be executed automatically.

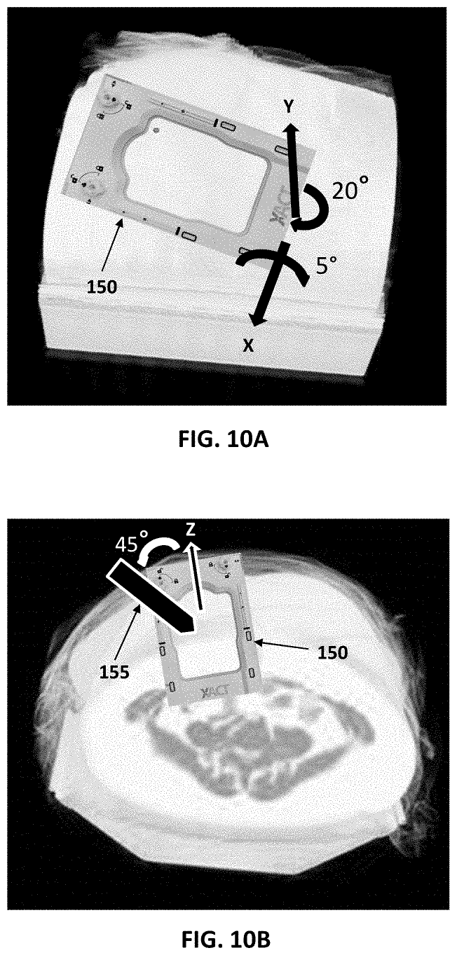

FIG. 10A schematically illustrates exemplary instructions given to the user, for example when an attachment frame 150 is used, regarding corrections to the position and orientation of the frame. As shown in FIG. 10A, the instructions are to rotate the attachment frame twenty (20) degrees clockwise about the Y axis and to rotate the frame (i.e., lift its distal end) five (5) degrees clockwise about the X axis. In some embodiments, the instructions may relate to the position and/or orientation of the end effector of the device. The instructions may be given as corrections relative to the current position or as absolute desired positioning relative to a known reference, e.g., an orientation angle about a certain axis, regardless of the current position. FIG. 10B schematically illustrates exemplary instructions given to the user, for example when an attachment frame 150, as well as an auxiliary positioning mechanism 155, such as the orienting member of the aiming jig shown in FIGS. 8A-8B), regarding corrections to the orientation of one or more components of the auxiliary positioning mechanism, which may simulate the device's end effector). As shown in FIG. 10B, the instructions are to rotate the one or more components of the auxiliary positioning mechanism 155 counter-clockwise such that it is oriented at 45 degrees relative to the Z axis. In some embodiments, the instructions may be a combination of instructions relating to the position and/or orientation of the device itself and instructions relating to the position and/or orientation of the end effector of the device.

At step 916, after the position and/or orientation correction instructions have been executed by the user, or automatically by the processor/controller, one or more new images of the region of interest are displayed. Then, steps 906 to 916 are repeated, until the actual position and orientation of the device is determined to be valid. In some embodiments, the number of iterations may be limited, i.e., steps 906-916 may be repeated only a limited number of times (iterations) and/or for a limited period of time. In some embodiments, if after the last iteration the actual position and orientation is still determined to be invalid, the user may be prompted to select a new entry point and/or adjust the marking of the target, and then steps 904 to 916 may be repeated, until the actual position and orientation of the device is determined to be valid. In some embodiments, the processor may present on the image-view/s a recommended range on the patient's skin for selecting the new entry point. The recommended range may be based on the position of the target and/or on the simulated position and orientation of the device. In some embodiments, steps 904-916 may be repeated with alternative entry points and/or adjusted target positions a limited number of iterations and/or for a limited period of time. If no valid position and orientation are found, the processor may notify the user that the medical device cannot be utilized in the procedure at hand.

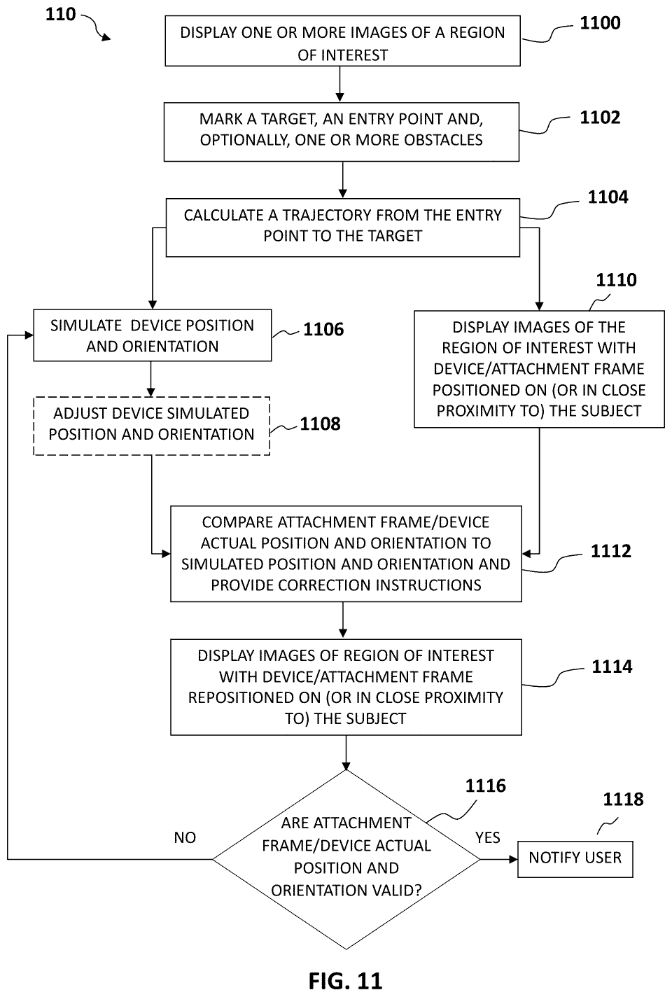

Reference is now made to FIG. 11, which illustrates a flowchart 110 showing the steps in another exemplary method for assisting the user to properly position the medical device on, or in proximally to, the patient's body. According to some embodiments, one or more of the steps shown in FIG. 11 may be optional and one or more of the steps may be repeated.

At step 1100, one or more images of the region of interest are displayed. In some embodiments, the images are obtained via imaging initiated immediately prior to the initiation of the medical procedure. In other embodiments, the images are obtained from the medical records of the subject, such as images taken days, or even weeks, prior to the execution of the medical procedure.

At step 1102, the target and the entry point and, optionally, obstacles, are marked/defined on the displayed image/s, for example, on one or more image-views generated from a set of images (or “slices”, or “image-frames”). Such image-views may be, for example, image-views pertaining to different planes or orientations (e.g., axial, sagittal, coronal, pseudo axial, pseudo sagittal, pseudo coronal, etc.) or other created views (e.g., trajectory view, tool view, 3D view, etc.). The marking may be done by the user (or based on user inputs) or automatically by the processor, with the user being required, optionally, to confirm and/or adjust the processor's proposed markings. In the latter case, the target (and optionally obstacle/s) and/or optimal entry point may be identified/suggested using known image processing techniques based on the obtained images of the region of interest and/or on data obtained from previous similar procedures, using machine learning and/or deep learning capabilities.

At step 1104, a trajectory from the entry point to the target is calculated. In case obstacles were marked on the initial image/s, the trajectory should be calculated such that it avoids the obstacles. Although a linear trajectory is generally preferred, a linear trajectory may not always be possible to plan, due to the physical location of the target, the presence of obstacles, etc., thus the planned trajectory may have a certain degree of curvature, which may be limited by a maximal allowable curvature level. In some embodiments, a calculated trajectory is considered valid if its curvature does not exceed a predetermined threshold. In some embodiments, the trajectory is calculated based on the displayed images of the region of interest and the marked locations of the entry point, target and obstacle/s and/or on data obtained from previous similar procedures using machine learning and/or deep learning capabilities.

At step 1106, the position and orientation of the medical device on the subject's body, or in proximity thereof, are simulated and displayed on the monitor using a virtual medical device/robot. In some embodiments, the simulated position and orientation are determined arbitrarily. In some embodiments, the virtual device may be displayed on the image-view/s at an arbitrary position and the user can then move and/or rotate the virtual device and determine the simulated position and orientation of the medical device relative to the subject's body (“manual positioning option”). In some embodiments, the virtual device may be displayed on the image-view/s at a position and orientation which are based on the processor's calculations (“automatic positioning option”). The processor's recommendation may be based on the displayed images of the region of interest and the calculated trajectory and/or on data obtained from previous similar procedures, using machine learning and/or deep learning algorithms. In some embodiments, the user can choose between the manual positioning option and the automatic positioning option. In some embodiments, the simulated position and orientation in the automatic positioning option may be based, inter alia, on scanning/registration limitations, parameters relating to the patient's body (e.g., body shape, etc.), user configurable restrictions (e.g., device facing direction), device setup considerations (e.g., avoiding target being directly under the device), the “squeezing” effect that attaching the device and/or the attachment frame has on the patient's body, all as described hereinabove in relation to step 306 of FIG. 3. In the manual positioning option, according to some embodiments, the user's input may be provided by means of “drag and drop”, i.e., the user may move and/or rotate the virtual device displayed on the monitor using the user interface, such as a computer mouse, a joystick or his/her own fingers (in case the monitor is a touch-screen), and release his/her “grip” of the virtual device, once the desired position and orientation are achieved. In some embodiments, the user interface may include virtual buttons for moving the virtual device in each of the X, Y and Z axis, separately or simultaneously (for example, dx, dy and dz buttons), and/or rotating the virtual device about each of the X, Y and Z axis, separately or simultaneously. In some embodiments, simulating the device's position and orientation may include adjusting the patient's pose, by rotating the image, to simulate different patient poses, such as prone, supine, decubitus etc. In the automatic positioning option, the image may be automatically rotated, should the processor's calculations determine that a different patient pose would be preferable for the specific procedure. In the manual positioning option, the user may rotate the image and simulate different device positions on the rotated image/s.

At optional step 1108, if required, the simulated position and orientation of the medical device on the subject's body, or in proximity thereof, are adjusted. In the automatic positioning option, the adjustment may be based on user input, e.g., using a “drag and drop” method and/or virtual buttons, as described hereinabove. In some embodiments, step 1108 may include adjustments executed by the processor. Automatic adjustment (e.g., for fine-tuning) may be required in the manual positioning option, after the user has determined the simulated position and orientation of the device and/or in the automatic positioning option, if after the processor has determined the simulated position and/or orientation of the device, the simulated position and orientation was adjusted by the user, as described hereinabove.

In some embodiments, in which the one or more images are obtained via imaging initiated immediately prior to the initiation of the medical procedure, steps 1102-1108 may be executed immediately prior to the medical procedure. In some embodiments, in which the one or more images are obtained from the medical records of the patient, steps 1102-1108 may be executed in advance, e.g., even before the patient arrives at the hospital/clinic for the procedure, and saved in the system's memory, or a cloud-based storage, until the time of the procedure. In some embodiments, steps 1102-1108 may be available to the user (e.g., physician) as a stand-alone software application that can communicate with the clinical software application (i.e., the software application used to control the insertion and steering of the medical instrument), or the output of which can be integrated into the clinical SW application, either automatically or manually by the user.

At step 1110, one or more new images of the region of interest are displayed, after the user has positioned the attachment frame and/or the medical device on the subject, or in close proximity thereto, based on his/her knowledge and experience. In case an attachment frame is utilized, the one or more images may be obtained after the attachment frame has been positioned on the subject's body, but before coupling the medical device to the frame. Further, the one or more images may be obtained after an auxiliary positioning mechanism (or device), for example the orienting member of the aiming jig described in FIGS. 8A-8B has been positioned and rotated to the orientation which the physician estimates, based on his knowledge and experience, will align the tip of the medical instrument with the insertion point and at an angle which will enable the instrument to be steered by the medical device until it reaches the target.

In some embodiments, in which the one or more images displayed in step 1100 are obtained from the medical records of the subject, and steps 1102 to 1108 are executed in advance, step 1110 is executed after step 1108. Alternatively, when the one or more images are obtained via imaging initiated immediately prior to the initiation of the medical procedure, steps 1106 and 1108 may be executed simultaneously with step 1110, or immediately thereafter, such that the simulation of the device's position and orientation (and, optionally, adjustments thereto) are executed on the images displayed at step 1110.

At step 1112, the actual position and orientation of the medical device and/or of the attachment frame is compared to the (final) simulated position and orientation. . In some embodiments, prior to comparing the actual position and orientation of the medical device and/or attachment frame to the simulated position and orientation of the medical device, the validity of the simulated position and orientation are determined, such that the actual position and orientation of the medical device and/or attachment frame is compared to the simulated position and orientation only if the simulated position and orientation are determined to be valid. In some embodiments, if the simulated position and orientation are determined to be invalid, a new simulated position and orientation of the device is generated, and then the validity of the new simulated position and orientation is determined prior to comparing the actual position and orientation to the new simulated position and orientation. In some embodiments, the calculation of new simulated position and orientation may be limited to a set time period and/or to a predetermined number of iterations. In some embodiments, if after the last iteration the simulated position and orientation is still determined to be invalid, the user may be prompted to select a new entry point and/or adjust the marking of the target. In some embodiments, if the actual position and orientation deviates from the simulated position and orientation instructions as to how to correct the current positioning so that it matches the simulated positioning are provided. In some embodiments, the instructions may relate to the position and/or orientation of the device (or attachment frame) itself, i.e., how (which direction) and how much (distance/angles) to move and/or rotate the device. In some embodiments, the instructions may relate to the position and/or orientation of the end effector of the device. In some embodiments, the instructions may be a combination of instructions relating to the position and/or orientation of the device itself and instructions relating to the position and/or orientation of the end effector of the device. The instructions may be given as corrections relative to the current position and orientation or as absolute desired positioning relative to a known reference, e.g., an orientation angle about a certain axis, regardless of the current position.

In some embodiments, an auxiliary positioning mechanism may be used to assist the user in applying the correction instructions provided by the processor to the position and/or orientation of the medical device as. The auxiliary positioning mechanism may be part of, or provided with, the automated medical device or the attachment frame. In some embodiments, the auxiliary positioning mechanism may be a stand-alone device. In some embodiments, the auxiliary positioning mechanism/device may be configured for manual use by the user, i.e., the user may be required to move/adjust certain elements of the auxiliary positioning device according to the provided correction instructions. In some embodiments, the auxiliary positioning mechanism/device may be automatic, i.e., it may be controllable by the processor/controller, such that the provided correction instructions may be executed automatically.

At step 1114, after the position and/or orientation correction instructions have been executed by the user, or automatically by the processor/controller, one or more new images of the region of interest are displayed, showing the repositioned attachment frame and/or the medical device.

At step 1116, it is determined if the current (corrected) position and orientation of the medical device (or the attachment frame) is valid, i.e., if the current position and orientation of the device and, optionally, also of the auxiliary positioning mechanism/device, as set by the user prior to the execution of step 1100, ensure alignment of the tip of the instrument with the insertion point and at the desired insertion angle, such that by the tip following the planned trajectory, it will reach the target (based on the position of the target in the image/s). In some embodiments, in order to determine if the simulated position and orientation of the medical device is valid, it may be required to verify that the trajectory, as calculated in step 1104, is still valid. In some embodiments, parameters pertaining to “robot feasibility” may be checked at step 1116, for example that the angles required from the end effector are within its feasible rotation range, etc. Additional parameters may be positioning limitations which relate to registration limits, such as maximal orientation angles of the medical device about one or more of the X, Y and Z axes. Additional parameters may be user configurable parameters, such as maximal distance of the device from the patient's skin surface, avoiding positioning of the robot directly above the target, etc.

If it is determined that the current position and orientation of the device is valid, then in step 1118, the user is notified. In some embodiments, the notification may be in an active form, i.e., a notification such as “Position Valid” may be displayed on the monitor and/or a similar audio notification may be provided via speakers. In some embodiments, the notification may be in a passive form, i.e., the procedure flow will automatically continue to the next step (e.g., if the medical device is attached to the patient's body, either directly or using an attachment frame, the user will be instructed to steer the instrument tip to the entry point), indicating that the current position and orientation of the device (or the attachment frame) has been determined as valid.

If it is determined that the current position and orientation of the device and/or the calculated trajectory are invalid, then steps 1106 to 1116 are repeated, until the actual position and orientation of the device is determined to be valid. In some embodiments, the number of iterations may be limited. In some embodiments, steps 1106-1116 may be repeated for a limited period of time. In some embodiments, if after the last iteration the actual position and orientation and/or the calculated trajectory are still determined to be invalid, the user may be prompted to select a new entry point and/or adjust the marking of the target, and then steps 1104 to 116 may be repeated, until the actual position and orientation of the device is determined to be valid. In some embodiments, the processor may recommend to the user, for example by marking on the image-view/s, a range/area on the patient's skin for selecting the new entry point. The recommended range may be based on the position of the target and/or on the simulated position and orientation of the device. In some embodiments, steps 1104-1116 may be repeated with alternative entry points and/or adjusted target positions a limited number of iterations and/or for a limited period of time. If no valid position and orientation are found, the processor may notify the user that the medical device cannot be used for the procedure at hand.

Although particular implementations have been disclosed herein in detail, this has been done by way of example for purposes of illustration only and is not intended to be limiting with respect to the scope of the appended claims, which follow. In particular, it is contemplated that various substitutions, alterations, and modifications may be made without departing from the spirit and scope of the disclosure as defined by the claims. Other aspects, advantages, and modifications are considered to be within the scope of the following claims. The claims presented are representative of the implementations and features disclosed herein. Other unclaimed implementations and features are also contemplated. Accordingly, other implementations are within the scope of the following claims.

It is to be understood that although some examples used throughout this disclosure relate to positioning an automated insertion device relative to the body of a subject, the disclosed devices, systems and methods are not limited for use with insertion devices alone and may be used with any medical device that is intended for positioning on, or in close proximity to, the subject's body.

Further, it is to be understood that although some examples used throughout this disclosure which relate to an automated insertion device, refer to insertion of a needle into a subject's body, this is done for simplicity reasons alone, and the scope of this disclosure is not limited to devices for insertion of a needle alone, but may include devices for insertion of any medical instrument intended to be inserted into a subject's body for diagnostic and/or therapeutic purposes, including needles, ports, introducers, probes (e.g., ablation probes), catheters (e.g., drainage needle catheters), cannulas, surgical tools, fluid delivery tools, or any other medical insertable tool.

In some embodiments, the term “attachment” of a medical device to a subject's body is to be interpreted as including both direct attachment to the body and attachment to the body via an intermediary element, such as an attachment frame, a cushion, etc.

In some embodiments, the term “positioning” of a medical device is to be interpreted as setting both the position and the orientation of the device. Further, in some embodiments, “position” may refer generally to both the position and the orientation of the device.

The terms “image”, “image frame”, “scan” and “slice” may be used interchangeably throughout this disclosure. The terms “medical instrument” and “medical tool” may be used interchangeably throughout this disclosure.

The terms “user”, “doctor”, “physician”, “clinician”, “technician”, “medical personnel” and “medical staff” are used interchangeably throughout this disclosure and may refer to any person taking part in the performed medical procedure.

In the description and claims of the application the expression “at least one of A and B”, (e.g. wherein A and B are elements, method steps, claim limitations, etc.) is equivalent to “only A, only B, or both A and B”. In particular, the expressions “at least one of A and B”, “at least one of A or B”, “one or more of A and B”, and “one or more of A or B” are interchangeable.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs.

Unless specifically stated otherwise, as apparent from the disclosure, it is appreciated that, according to some embodiments, terms such as “processing”, “computing”, “calculating”, “determining”, “estimating”, “assessing”, “gauging” or the like, may refer to the action and/or processes of a computer or computing system, or similar electronic computing device, that manipulate and/or transform data, represented as physical (e.g. electronic) quantities within the computing system's registers and/or memories, into other data similarly represented as physical quantities within the computing system's memories, registers or other such information storage, transmission or display devices.

The embodiments described in the present disclosure may be implemented in digital electronic circuitry, or in computer software, firmware or hardware, or in combinations thereof. The disclosed embodiments may be implemented as one or more computer programs, i.e., one or more modules of computer program instructions, encoded on computer storage medium for execution by, or to control the operation of, one or more data processing apparatus. Alternatively or in addition, the computer program instructions may be encoded on an artificially generated propagated signal, for example, a machine-generated electrical, optical or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of any one or more of the above. Furthermore, while a computer storage medium is not a propagated signal, a computer storage medium can be a source or destination of computer program instructions encoded in an artificially generated propagated signal. The computer storage medium can also be, or be included in, one or more separate physical components or media (for example, multiple CDs, disks, or other storage devices).

The operations described in the present disclosure can be implemented as operations performed by a data processing apparatus on data stored on one or more computer-readable storage devices or received from other sources.

The term “data processing apparatus” as used herein may encompass all types of apparatus, devices, and machines for processing data, including by way of example a programmable processor, a computer, a system on a chip/s, or combinations thereof. The data processing apparatus can include special purpose logic circuitry, for example, an FPGA (field programmable gate array) or an ASIC (application specific integrated circuit). The apparatus can also include, in addition to hardware, code that creates an execution environment for the computer program in question, for example, code that constitutes processor firmware, a protocol stack, a database management system, an operating system, a cross-platform runtime environment, a virtual machine, or combinations thereof. The apparatus and execution environment can realize various different computing model infrastructures, such as web services, distributed computing and grid computing infrastructures.

A computer program (also referred to as a program, software, software application, script or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A computer program can be stored in a portion of a file that holds other programs or data, in a single file dedicated to the program in question, or in multiple coordinated files (for example, files that store one or more modules, sub programs or portions of code). A computer program can be deployed to be executed on one computer or on multiple computers that are located at one site or distributed across multiple sites and interconnected by a communication network.

The processes and logic flows described herein can be performed by one or more programmable processors, executing one or more computer programs to perform actions by operating on input data and generating output. The processes and logic flows can also be performed by, and an apparatus can also be implemented as, special purpose logic circuitry, for example, an FPGA or an ASIC. Processors suitable for the execution of a computer program include both general and special purpose microprocessors, and any one or more processors of any type of digital computer. Generally, a processor will receive instructions and data from a read-only memory or a random access memory or both. The essential elements of a computer are a processor for performing actions in accordance with instructions and one or more memory devices for storing instructions and data. A computer may, optionally, also include, or be operatively coupled to receive data from or transfer data to, or both, one or more mass storage devices for storing data, for example, magnetic, magneto optical discs, or optical discs. Moreover, a computer can be embedded in another device, for example, a mobile phone, a tablet, a personal digital assistant (PDA, a game console, a Global Positioning System (GPS) receiver, or a portable storage device (for example, a USB flash drive). Devices suitable for storing computer program instructions and data include all forms of non-volatile memory, media and memory devices, including semiconductor memory devices, for example, EPROM, EEPROM and flash memory devices; magnetic discs, for example, internal hard discs or removable discs; magneto optical discs; CD ROM and DVD-ROM discs; solid state drives (SSDs); and cloud-based storage. The processor and the memory can be supplemented by, or incorporated in, special purpose logic circuitry.

The processes and logic flows described herein may be performed in whole or in part in a cloud computing environment. For example, some or all of a given disclosed process may be executed by a secure cloud-based system comprised of co-located and/or geographically distributed server systems. The term “cloud computing” is generally used to describe a computing model which enables on-demand access to a shared pool of computing resources, such as computer networks, servers, software applications, and services, and which allows for rapid provisioning and release of resources with minimal management effort or service provider interaction.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. The methods of the disclosure may include a few of the steps described or all of the steps described. No particular step in a disclosed method is to be considered an essential step of that method, unless explicitly specified as such.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

1.-37. (canceled)

38. A method of assisting a user in positioning an automated medical device on, or in close proximity to, a body of a subject, comprising:
displaying one or more images of a region of interest within the body of the subject;
defining on the one or more images a target and an entry point;
calculating a trajectory for a medical instrument from the entry point to the target;
simulating on the one or more images a position and an orientation of the automated medical device on, or in close proximity to, the body of the subject;
determining if the simulated position and orientation of the automated medical device is valid; and
if the simulated position and orientation of the automated medical device and the calculated trajectory are determined to be valid, providing positioning instructions to the user as to the positioning of the automated medical device on, or in close proximity to, the body of the subject.

39. The method of claim 1, wherein if the simulated position and orientation of the automated medical device are determined to be invalid, the method comprises prompting the user to define a new entry point and/or a new target on the one or more images.

40. The method according to claim 38, wherein if the simulated position and orientation of the automated medical device are determined to be invalid, the method comprises
simulating on the one or more images one or more additional positions and orientations of the automated medical device,
determining if at least one of the one or more additional simulated positions and orientations of the automated medical device is valid, and
providing positioning instructions to the user as to the positioning of the automated medical device on, or in close proximity to, the body of the subject.

41. The method of claim 38, wherein if the simulated position and orientation of the automated medical device are determined to be invalid, the method further comprises recommending to the user an alternative simulated position and orientation of the automated medical device.

42. The method according to claim 38, wherein determining if the simulated position and orientation of the automated medical device are valid comprises determining if the simulated position and orientation ensure alignment of a tip of the medical instrument with the entry point at an insertion angle that enables inserting and steering of the medical instrument according to the calculated trajectory, from the entry point to the targetor wherein determining if, for the simulated position and orientation of the automated medical device, the calculated trajectory is valid; , wherein determining if the calculated trajectory is valid comprises determining if a curvature of the calculated trajectory exceeds a predetermined threshold.

43. The method according to claim 38, wherein determining if the simulated position and orientation of the automated medical device are valid comprises determining if rotation angles required from an end effector of the automated medical device are within a feasible rotation range for the end effector.

44. The method according to claim 38, comprising defining on the one or more images one or more obstacles to be avoided by the medical instrument, and wherein the automated medical device is an automated insertion device configured to insert and steer the medical instrument toward the target according to the calculated trajectory.

45. The method according to claim 38, wherein simulating a position and an orientation of the automated medical device on the one or more images comprises displaying a virtual medical device on the one or more images; and/or wherein simulating a position and an orientation of the automated medical device on the one or more images is executed automatically by means of at least one processor using image processing techniques.

46. The method according to claim 45, wherein automatically simulating a position and an orientation of the automated medical device is executed using one or more machine learning and/or deep learning algorithms.

47. The method according to claim 38, wherein simulating a position and an orientation of the automated medical device comprises receiving user input regarding a position and an orientation of a virtual medical device displayed on the one or more images.

48. The method according to claim 38, wherein providing positioning instructions to the user comprises
at least one of displaying the positioning instructions on a monitor and providing audio positioning instructions, and
providing positioning instructions as to the positioning of an attachment apparatus configured for securing to the body of the subject and for coupling the automated medical device thereto, on the body of the subject; , wherein the positioning instructions provided to the user comprise instructions to one or more of move, rotate, elevate or tilt the automated medical device.

49. A system for assisting a user in positioning an automated medical device on, or in close proximity to, a body of a subject, comprising:
at least one processor configured to execute the method of claim 1; and
a monitor configured to display at least one of: one or more images, a calculated trajectory and a simulated position and orientation of the automated medical device

50. The system according to claim 49, further comprising a user interface.

51. The system according to claim 49, wherein the automated medical device is configured to be attached to the body of the subject using an attachment apparatus;
wherein the attachment apparatus comprises one or more of a parallel lifting member configured to elevate the automated medical device relative to a surface of the body of the subject and an angular lifting member configured to tilt the automated medical device relative to the surface of the body of the subject

52. The system according to claim 49, further comprising an auxiliary positioning mechanism configured to assist the user in at least one of positioning and orienting the automated medical device based on the provided positioning instructions.

53. The system of claim 52, wherein the auxiliary positioning mechanism comprises an orienting member configured to simulate one or more of the medical instrument and an end effector of the automated medical device.

54. The system according to claim 52, wherein the auxiliary positioning mechanism comprises an electromechanical mechanism and is configured to be controlled by the processor to automatically execute the positioning instructions.

55. The system according to claim 52, wherein the auxiliary positioning mechanism is a stand-alone device.

56. The system according to claim 52, wherein the auxiliary positioning mechanism is part of, or couplable to, an attachment apparatus or an aiming apparatus, the attachment apparatus being configured for securing to the body of the patient and for receiving the automated medical device thereon, and the aiming apparatus being configured for removably coupling to the attachment apparatus and for assisting in ensuring alignment between a tip of the medical instrument and an entry point marked on the body of the patient.

57. A system for assisting a user in positioning an automated medical device on, or in close proximity to, a body of a subject, comprising:
at least one processor configured to execute a method of assisting a user in positioning an automated medical device on, or in close proximity to, a body of a subject, the method comprising:
displaying one or more first images of a region of interest in the body of the subject, the one or more images showing one or more of the automated medical device and an attachment apparatus configured to be secured to the body of the patient and to receive the automated medical device thereon, positioned on the body of the subject at an initial position and orientation;
defining on the one or more images a target and an entry point;
calculating a trajectory for the medical instrument from the entry point to the target;
simulating on the one or more images a position and an orientation of the automated medical device on, or in close proximity to, the body of the subject;
comparing the actual position and orientation of the one or more of the automated medical device and the attachment frame to the simulated position and orientation of the automated medical device;
if the actual position and orientation of the one or more of the automated medical device and the attachment frame deviate from the simulated position and orientation of the automated medical device, providing correction instructions to the user as to the required correction to the actual position and orientation of the one or more of the automated medical device and the attachment frame;
displaying one or more second images of the region of interest, the one or more second images showing the one or more of the automated medical device and the attachment frame positioned on the body of the subject at a corrected position and orientation;
determining if the corrected position and orientation of the one or more of the automated medical device and the attachment frame are valid; and
if the corrected position and orientation of the one or more of the automated medical device and the attachment frame are determined to be valid, notifying the user; and
a monitor configured to display at least one of: one or more images, a calculated trajectory and a simulated position and orientation of the automated medical device.
